(19) United States
(12) Patent Application Publication
(10) Pub. No.: US 2010/0062973 A1
(43) Pub. Date: Mar. 11, 2010

(54) Production of bioactive glycoproteins from inactive starting material

(75) Inventors: Ronald Frank, Meine-Grassel (DE); Harald S. Conradt, Braunschweig (DE); Eckart Grabenhorst, Braunschweig (DE); Norbert Zander, Meine (DE)

Correspondence Address: FISH & RICHARDSON P.C., PO BOX 1022, MINNEAPOLIS, MN, 55440-1022, US

(73) Assignee: FRESENIUS KABI DEUTSCHLAND GmbH, Bad Homburg (DE)

(21) Appl. No.: 11/908,325

(22) PCT Filed: Mar. 9, 2006
(86) PCT No.: PCT/EP2006/002179

§ 371 Date: Aug. 19, 2009

Related U.S. Application Data
(60) Provisional application No. 60/660,629, filed on Mar. 11, 2005.

(51) Int. Cl. A61K 47/48; C07K 14/00; C12P 21/00

(52) U.S. Cl. 514/8; 530/395; 435/68.1

(57) ABSTRACT

A method for preparing a conjugate comprising a glycoprotein and a polymer or a derivative of said polymer, wherein the polymer is a hydroxyalkylstarch (HAS), the method comprising the steps a) reacting said glycoprotein with a modified polyol carrying attached thereto, by a covalent linkage, a functional group Z, in the presence of a transferase capable of catalysing formation of a covalent linkage between the glycoprotein and said modified polyol, yielding a glycoprotein covalently linked to a polyol carrying attached thereto, by a covalent linkage, at least one functional group Z, and b) reacting at least one functional group A of the polymer or the derivative thereof with the at least one functional group Z of the glycoprotein added to said glycoprotein during step a), and thereby forming a covalent linkage.

Erythropoietin is an acid glycoprotein hormone of approximately 34,000 D. Human erythropoietin is a 166 amino acid polypeptide that exists naturally as a monomer (Lin et al., 1985, PNAS 82, 7580-7584, EP 148 605 B2, EP 411 678 B2). The identification, cloning and expression of genes encoding erythropoietin are described, e.g., in U.S. Pat. No. 4,703,008. The purification of recombinant erythropoietin from cell culture medium that supported the growth of mammalian cells containing recombinant erythropoietin plasmids, is for example, is described in U.S. Pat. No. 4,667,016.

It is generally believed in this technical field that the biological activity of EPO in vivo mainly depends on the degree of sialic acids bound to EPO (see e.g. EP 428 267 B1). Theoretically, 14 molecules of sialic acid can be bound to one molecule EPO at the terminal ends of the carbohydrate side chains linked to N- and O-glycosylation sites. However, in the international EPO standard preparation (BRP-EPO standard batch II) which is a recombinantly expressed human molecule preparation from Chinese hamster Ovary cells (CHO), EPO isoforms are present that have 10-14 or 15 sialic acid residues. In order to guarantee proper in vivo biological activity of EPO the number of terminally sialic masked galactose residues of its complex-type N-glycan moieties is of importance.

The same holds true for other glycoproteins for use in pharmaceutical applications. The in vivo bioactivity of glycoproteins like IFN-B, antibodies, hcg, FSH, and LH is also thought to depend on the proper capping of their N- and O-Glycans with sialic acid.

In the case of EPO, highly sophisticated purification steps are necessary to obtain highly sialylated EPO preparations from recombinant sources which show a sufficient high in vivo biological activity. Therefore a significant proportion of the total EPO secreted by cells into the medium has to be separated from high activity forms of the final desired product. It would be of advantage to make use of undersialylated forms of EPO—and of undersialylated forms of other glycoproteins in general—which are usually discarded as not suitable for pharmaceutical applications.

The present invention describes a gentle method for how to render such undersialylated glycoprotein preparations useful for pharmaceutical applications. The use of enzymes to introduce functional groups into glycoproteins and the subsequent covalent HES-modification at the introduced functional groups yields modified glycoproteins with in vivo activities comparable with or even higher than that of the properly sialylated glycoprotein form. It is described herein, using human EPO as an example, that a preparation of EPO severely deficient in terminal sialic acid residues can be modified to yield a preparation comparable in in vivo bioactivity with a preparation containing a total of 14 sialic acids per molecule of EPO.

### DETAILED DISCLOSURE OF THE INVENTION

Therefore, the present invention relates to a method for preparing a conjugate comprising a glycoprotein and a polymer or a derivative of said polymer, wherein the polymer is a hydroxyalkylstarch (HAS), the method comprising the steps
a) reacting said glycoprotein with a modified polyol carrying attached thereto, by a covalent linkage, a functional group Z, in the presence of a transferase capable of catalysing formation of a covalent linkage between the glycoprotein and said modified polyol, yielding a glycoprotein covalently linked to a polyol carrying attached thereto, by a covalent linkage, at least one functional group Z, and
b) reacting at least one functional group A of the polymer or the derivative thereof with the at least one functional group Z of the glycoprotein added to said glycoprotein during step a), and thereby forming a covalent linkage.

In general, there are no specific restricions as to the functional groups A and Z with the proviso that a covalent linkage can be formed by reacting A and Z. The following groups A are, e.g., conceivable:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2 amino-thioalcohols;
1,2-aminoalcohols;
the amino group —NH₂ or derivatives of the amino groups comprising the structure unit —NH— such as aminoalkyl groups, aminoaryl group, aminoaralkyl groups, or alkarlyaminogroups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—; residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxylamines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
—NH—NH₂, or —NH—NH—;
—NO₂;
the nitril group;
carbonyl groups such as the aldehyde group or the keto group or a hemiacetal group;
the carboxy group;
the —N═C═O group or the —N═C═S group;
vinyl halide groups such as the vinyl iodide or the vinyl bromide group or triflate;
—C≡C—H
—(C═NH₂Cl)-OAlkyl
groups —(C═O)—CH₂-Hal is Cl, Br, or I;
—CH═CH—SO₂—;
a disulfide group comprising the structure —S—S—;
the group

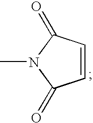

the group

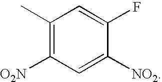

Accordingly, the functional group Z is a group capable of forming a covalent chemical linkage with functional group A, preferably with a functional group A which is one of the above-mentioned functional groups. More preferably, Z is selected from the above-mentioned groups.

Preferably, step b) is conducted by reacting at least one functional group A of the polymer or the derivative thereof with at least one functional group Z of the glycoprotein, which was modified by introduction of group Z in step a), and thereby forming a covalent linkage, wherein Z is selected from the group consisting of an amino group, a thiol group, an aldehyde group, a hemiacetal group, a keto group, a maleimido group, and a thioester group.

More preferably, step b) is conducted by reacting at least one functional group A of the polymer or the derivative thereof with at least one functional group Z of the glycoprotein added to said glycoprotein during step a), and thereby forming a covalent linkage, wherein Z is selected from the group consisting of an amino group, a thiol group, an aldehyde group, a hemiacetal group, a keto group, a maleimido group, and an thioester group,
wherein, in case Z is an aldehyde group or a keto group, A comprises an amino group forming said linkage with Z,
wherein, in case Z is an amino group, A is selected from the group consisting of a reactive carboxy group and an aldehyde group, a keto group or a hemiacetal group,
wherein, in case Z is a maleimido group, A comprises an thiol group forming said linkage with Z,
   wherein, in case A is an aldehyde group, a keto group or a hemiacetal group, the method further comprises introducing A in the polymer to give a polymer derivative
      by reacting the polymer with an at least bifunctional compound, one functional group of which reacts with the polymer and at least one other functional group of which is an aldehyde group, a keto group or a hemiacetal group, or is a functional group which is further chemically modified to give an aldehyde group, a keto group or a hemiacetal group, or
      by oxidizing the polymer to give at least one, in particular at least two aldehyde groups, or
   wherein, in case A is a reactive carboxy group, the method further comprises introducing A in the polymer to give a polymer derivative
      by selectively oxidizing the polymer at its reducing end and activating the resulting carboxy group, or
      by reacting the polymer at its non-oxidized reducing end with a carbonic diester, or
wherein, in case Z is a thiol group and A comprises
   a maleimido group or
   a halogenacetyl group
      forming said linkage with Z.

The enzymatic transfer of functional group Z to the glycoprotein has the advantage of being very gentle and avoiding oxidative and/or acidic conditions, which could lead to oxidation of methionen residues and/or desamidation of glutamine/asparagines residues. Furthermore, such functional groups Z may be attached to glycoproteins, that are usually not present in proteins, thus making the subsequent reaction with the functional group A potentially very site specific.

The choice of functional groups Z attached to the glycoprotein and A attached to the hydroxyalkylstarch is such that they react with one another under gentle conditions. In particular,
in case Z is an aldehyde group or a keto group, A comprises an amino group forming said linkage with Z,
in case Z is an maleimido group, A comprises a thiol, and
in case Z is an amino group, A is selected from the group consisting of a reactive carboxy group and an aldehyde group, a keto group or a hemiacetal group.

The method of the invention further includes the introduction of the functional group A into the polymer to give a polymer derivative capable of reacting with the modified glycoprotein after addition of functional group Z in step a).

Thus, in case A is an aldehyde group, a keto group or a hemiacetal group, the method further comprises introducing A in the polymer to give a polymer derivative
by reacting the polymer with an at least bifunctional compound, one functional group of which reacts with the polymer and at least one other functional group of which is an aldehyde group, a keto group or a hemiacetal group, or is a functional group which is further chemically modified to give an aldehyde group, a keto group or a hemiacetal group, or
by oxidizing the polymer to give at least one, in particular at least two aldehyde groups, or

Thus, in case A is a reactive carboxy group, the method further comprises introducing A in the polymer to give a polymer derivative
by selectively oxidizing the polymer at its reducing end and activating the resulting carboxy group, or
by reacting the polymer at its non-oxidized reducing end with a carbonic diester.

In case Z is a thiol group, A comprises a maleimido group or a halogenacetyl group forming said linkage with Z. The introduction of a thiol group Z during step a) is preferred in those cases, where the glycoprotein itself has no free thiol groups available for reaction with group A. This is, for example, often the case in secreted glycoproteins, where often all cysteine residues are engaged in intraprotein cystein bridges.

In case Z is a maleimido group, A comprises a thiol group forming said linkage with Z. The introduction of a maleimido group Z during step a) is a preferred embodiment, since the glycoprotein itself usually has no such functional groups available for a reaction with A.

In case Z is an amino group, A comprises a reactive carboxy group, an aldehyde group, a keto group or a hemiacetal group. The introduction of an amino group, in particular wherein Z is a hydroxylamino group or a hydrazido group, during step a) is a preferred embodiment, since the glycoprotein itself usually has no such functional groups available for a reaction with A.

A further advantage of the method of the invention is the high yield of its individual steps and of the overall method. Therefore, the invention also relates to a method as described above, wherein at least 50%, preferably at least 75%, more preferably at least 90%, of the modified glycoprotein obtained from step a) is converted to the product conjugate during step b). The product conjugate comprises the input glycoprotein and a polymer or a polymer derivative. The polymer or polymer derivative may be attached to the glycoprotein via the covalent bond formed between group Z of the modified polyol and group A of the polymer or polymer derivative. Preferably, the polymer or polymer derivative is exclusively attached to the glycoprotein via the covalent bond formed between group Z of the modified polyol and group A of the polymer or polymer derivative, that is, the polymer or the polymer derivative react only with the functional group provided by addition of the modified polyol to the glycoprotein.

Therefore, the invention also relates to a method as described above, wherein at least 50%, preferably at least 75%, more preferably at least 90%, of the starting—that is unmodified—glycoprotein is converted in step a) to a modified glycoprotein—that is a glycoprotein covalently linked to a polyol carrying attached thereto, by a covalent linkage, at least one functional group Z.

The polyol substrates to be transferred to the glycoproteins and the transferases used in the transferase reaction may be all polyols and all transferases disclosed in WO03/031464. A modified polyol as used herein is any polyol that is modified, but still a suitable substrate of a glycosyltransferase. In a preferred embodiment, the modified polyol is a modified sugar nucleotide, in particular a modified fucose-, glucose-, mannose-, N-Acetylglucosamin-, N-Acetygalactosamin- or galactose-nucleotide, more preferably CMP-NeuAc, GDP-Man, UDP-GlcNAc, UDP-Gal-NAc, UDP-Glc or GDP-Fuc.

The modified polyols, and in particular the modified sugar nucleotides, may carry the functional group Z directly attached to a carbon atom of the polyol backbone (e.g. a thiol group attached to C6 of a glucose residue), or the functional group Z may be attached via a linker molecule. Among others, the linker may be an optionally substituted, linear, branched and/or cyclic hydrocarbon residue. Generally, the hydrocarbon residue has up to 60, preferably up to 40, more preferably up to 20, more preferably up to 10, more preferably up to 6 and especially preferably up to 4 carbon atoms. If heteroatoms are present, the separating group comprises generally from 1 to 20, preferably from 1 to 8, more preferably 1 to 6, more preferably 1 to 4 and especially preferably from 1 to 2 heteroatoms. As heteroatom, O is preferred. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment of the present invention, the linker is a linear hydrocarbon chain having 4 carbon atoms. According to another preferred embodiment of the present invention, the linker is a linear hydrocarbon chain having 4 carbon atoms and at least one, preferably one heteroatom, particularly preferably an oxygen atom. Such modified and linker-modified polyols can be prepared by organo-chemical synthesis, for example as described in “Carbohydrates in Chemistry and Biology part I, Vols. 1+2, B. Ernst, G. W. Hart and P. Sinay eds. Published 2000, Whiley-VCH Weinheim-New York-Chichester-Brisbane-Toronto, ISBN 3-527-29511-9.

Preferred transferases useful in step a) of the method of the invention are glycosyltransferases of the EC class 2.4.1, in particular wherein the tranferase is selected from the group consisting of β-1,4-galactosyltransferase, β-1,3-galactosyltransferase, α-1,3-galactosyltransferase, GlcNAc-transferase, mannosyltranferase, glucosyltransferase, fucosyltransferase and sialyltransferase.

In the context of the present invention, the term “glycosylated protein” or “glycoprotein”, i.e. a protein having a “carbohydrate side chain” refers to proteins comprising carbohydrate moieties such as hydroxyaldehydes or hydroxyketones as well as to chemical modifications thereof (see Römpp Chemielexikon, Thieme Verlag Stuttgart, Germany, 9th edition 1990, Volume 9, pages 2281-2285 and the literature cited therein). Furthermore, it also refers to derivatives of naturally occurring carbohydrate moieties like, galactose, N-acetylneuramic acid, and N-acetylgalactosamine) and the like.

Preferred glycoproteins which can be conjugated according to the invention can be characterized as follows:

Erythropoietin: The EPO can be of any human (see e.g. Inoue, Wada, Takeuchi, 1994, An improved method for the purification of human erythropoietin with high in vivo activity from the urine of anemic patients, Biol Pharm Bull. 17(2), 180-4; Miyake, Kung, Goldwasser, 1977, Purification of human erythropoietin, J Biol Chem., 252(15), 5558-64) or another mammalian source and can be obtained by purification from naturally occurring sources like human kidney, embryonic human liver or animal, preferably monkey kidney. Furthermore, the expression “erythropoietin” or “EPO” encompasses also an EPO variant wherein one or more amino acids (e.g. 1 to 25, preferably 1 to 10, more preferred 1 to 5, most preferred 1 or 2) have been exchanged by another amino acid and which exhibits erythropoietic activity (see e.g. EP 640 619 B1). The measurement of erythropoietic activity is described in the art (for measurement of activity in vitro see e.g. Fibi et al., 1991, Blood, 77, 1203 ff; Kitamura et al., 1989, J. Cell Phys., 140, 323-334; for measurement of EPO activity in vivo see Ph. Eur. 2001, 911-917; Ph. Eur. 2000, 1316 Erythropoietini solutio concentrata, 780-785; European Pharmacopoeia (1996/2000); European Pharmacopoeia, 1996, Erythropoietin concentrated solution, Pharmaeuropa., 8, 371-377; Fibi, Hermentin, Pauly, Lauffer, Zettlmeissl, 1995, N- and O-glycosylation muteins of recombinant human erythropoietin secreted from BHK-21 cells, Blood, 85(5), 1229-36; (EPO and modified EPO forms were injected into female NMRI mice (equal amounts of protein 50 ng/mouse) at day 1, 2 and 3 blood samples were taken at day 4 and reticulocytes were determined)). Further publications where tests for the measurement of the activity of EPO are described Barbone, Aparicio, Anderson, Natarajan, Ritchie, 1994, Reticulocytes measurements as a bioassay for erythropoietin, J. Pharm. Biomed. Anal., 12(4), 515-22; Bowen, Culligan, Beguin, Kendall, Villis, 1994, Estimation of effective and total erythropoiesis in myelodysplasia using serum transferrin receptor and erythropoietin concentrations, with automated reticulocyte parameters, Leukemi, 8(1), 151-5; Delorme, Lorenzini, Giffin, Martin, Jacobsen, Boone, Elliott, 1992, Role o glycosylation on the secretion and biological activity of erythropoietin, Biochemistry, 31(41), 9871-6; Higuchi, Oh-eda, Kuboniwa, Tomonoh, Shimonaka, Ochi, 1992; Role of sugar chains in the expression of the biological activity of human erythropoietin, J. Biol. Chem., 267(11), 7703-9; Yamaguchi, Akai, Kawanishi, Ueda, Masuda, Sasaki, 1991, Effects of site-directed removal of N-glycosylation sites in human erythropoietin on its production and biological properties, J. Biol. Chem., 266(30), 20434-9; Takeuchi, Inoue, Strickland, Kubota, Wada, Shimizu, Hoshi, Kozutsumi, Takasaki, Kobata, 1989, Relationship between sugar chain structure and biological activity of recombinant human erythropoietin produced in Chinese hamster ovary cells, Proc. Natl. Acad. Sci. USA, 85(20), 7819-22; Kurtz, Eckardt, 1989, Assay methods for erythropoietin, Nephron., 51(1), 11-4 (German); Zucali, Sulkowski, 1985, Purification of human urinary erythropoietin on controlled-pore glass and silicic acid, Exp. Hematol., 13(3), 833-7; Krystal, 1983, Physical and biological characterization of erythroblast enhancing factor (EEF), a late acting erythropoetic stimulator in serum distinct from erythropoietin, Exp. Hematol., 11(1), 18-31.

HCG stimulates the ovaries to synthesize the steroids that are essential for the maintenance of pregnancy. It is a placental secreted heterodimer of a common alpha chain and a unique beta chain which confers biological specificity to thyrotropin, lutropin, follitropin and gonadotropin. It is produced by the first trimester placenta. It is commercially available under the names Novarel (Ferring) and Profasi (Serono). HCG is used as adjunctive therapy in the treatment of obesity. The beta chain contains two N-glycosylation sites and 4 O-glycosylation sites. HCG belongs to the glycoprotein hormones beta chain family. HCG is a hormone released by the placenta (“pregnancy hormone”) as well as various tumors, but locally produced and acting also within the testis and other tissues. It is a member of the glycoprotein hormone (GPH) family, the others being the pituitary hormones follicle stimulating hormone (FSH), luteinizing hormone (LH) and thyroid stimulating hormone (TSH). Each of these is a heterodimer consisting of a common alpha and a hormone-specific beta subunit. The subunits are partly homologous to each other, most predominantly in terms of tertiary structure. Recent elucidation of the crystal structure of hCG has revealed that all these subunits share the socalled cystin-knot structural motif with growth factors such as nerve (NGF), platelet-derived (PDGF), Transforming (TGFbeta) growth factor and others, that are otherwise unrelated to the GPH. The beta subunit of hCG is quite similar to that of LH (Lapthorn, A J, Harris, D. C., Littlejohn, A, Lustbader, J W, Canfield, R E, Machin, K J, Morgan, F J, Isaacs, N W: Crystal structure of human chorionic gonadotropin. Nature, 369, 455-461, 1994).

LH promotes spermatogenesis and ovulation by stimulating the testes and ovaries to synthesize steroids. It is secreted by the pituitary gland and is heterodimer of a common alpha chain and a unique beta chain which confers biological specificity to thyrotropin, lutropin, follitropin and gonadotropin. Defects in LHB are a cause of hypogonadism, a disease characterized by infertility and pseudohermaphroditism. LH belongs to the glycoprotein hormones beta chain family (Weisshaar G., Hiyama J., Renwick A. G. C., Nimtz M.; “NMR investigations of the N-linked oligosaccharides at individual glycosylation sites of human lutropin.”; Eur. J. Biochem. 195:257-268 (1991)).

FSH is a heterodimer of a common alpha chain and a unique beta chain which confers biological specificity to thyrotropin, lutropin, follitropin and gonadotropin It is commercially available under the names Gonal-F or Metrodin HP (Serono) and Puregon (Organon) and is used in the treatment of infertility in women with proven hypopituitarism or who have not responded to clomifene; or in superovulation treatment for assisted conception (such as in vitro fertilisation). Metrodin HP is also used in the treatment of hypogonadotrophic hypogonadism in men for the stimulation of spermatogenesis (Fujiki Y., Rathnam P., Saxena B. B.; “Studies on the disulfide bonds in human pituitary follicle-stimulating hormone.”; Biochim. Biophys. Acta 624:428-435 (1980) and Keene J. L., Matzuk M. M., Otani T., Fauser B. C. J. M., Galway A. B., Hsueh A. J. W., Boime I.; “Expression of biologically active human follitropin in Chinese hamster ovary cells.”; J. Biol. Chem. 264:4769-4775 (1989)).

Antibodies fusion proteins are known as therapeutic agents and from clinical trials e.g attempting to augment and potentiate the host defense systems against breast cancer. Combination of IL 2, IL 12, GM CSF and either an Fc part of human IgG or a single-chain variable domain (scFv) directed against a suitable target e.g mediate T cell immuno stimulation with the targeting specificity and ease of delivery of monoclonal antibodies been developed. Typically antibody fusion proteins have N-glycosylation sites at the Fc-moiety and may contain N- and O-glycosylation sites at the cytokine portion of the molecule (Antibody Fusion Proteins 312 pages Editor: Steven M. Chamow; Editor: Avi Ashkenazi; John Wiley & Sons, Inc).

An interleukin, especially interleukine 2 or 6 are produced by T-cells in response to antigenic or mitogenic stimulation, this protein is required for T-cell proliferation and other activities crucial to regulation of the immune response. Can stimulate B cells, monocytes, lymphokine-activated killer cells, natural killer cells, and glioma cells. Interleucine 2 is involved n a form of T-cell acute lymphoblastic leukemia (T-ALL) by a chromosomal translocation t(4;16)(q26;p13) which involves TNFRSF17 and IL2. It is commercial available under the name Proleucin (Chiron) and is used in patients with renal cell carcinoma or metastatic melanoma. J. Biol. Chem. 264:17368-17373 (1989).

Interleukin 6 is a cytokine with a wide variety of biological functions: it plays an essential role in the final differentiation of B-cells into Ig-secreting cells, it induces myeloma and plasmacytoma growth, it induces nerve cells differentiation, in hepatocytes it induces acute phase reactants J. Mol. Cell. Immunol. 4:203-211 (1989).

Granulocyte/macrophage colony-stimulating factors are cytokines that act in hematopoiesis by controlling the production, differentiation, and function of 2 related white cell populations of the blood, the granulocytes and the monocytes-macrophages. G-CSF induces granulocytes. The molecular weight of the signal cleaved mature protein is 19046 dalton. It contains a single O-glycosylation site. G-CSF is available under the names Neupogen or Granulokine (Amgen/Roche) and Granocyte (Rhone-Poulenc). G-CSF is used to treat neutropenia (a disorder characterized by an extremely low number of neutrophils in blood).

Interferons are cytokines that mediate antiviral, anti-proliferative and immuno-modulatory activities in response to viral infection and other biological inducers. The amino acid sequence of human interferon beta is given, e.g. in EP 0 218 825 A1. Useful commercial preparations of interferon beta are Avonex and Rebif (IFN beta 1a). Interferon beta 1a is produced by recombinant DNA technology using genetically engineered Chinese Hamster Ovary (CHO) cells into which the human interferon beta gene has been introduced. The amino acid sequence of IFN beta 1a is identical to that of natural fibroblast derived human interferon beta. Natural interferon beta and interferon beta 1a are glycosylated with each containing a single N-linked complex carbohydrate moiety at the Asn80. The interferon beta drugs are indicated for the treatment of relapsing remitting multiple sclerosis.

IFN alpha forms are naturally produced by monocytes/macrophages, lymphoblastoid cells, fibroblasts and a number of different cell types following induction by viruses, nucleic acids, glucocorticoid hormones, and other inductors. At least 23 different variants of IFN alpha are known. The individual proteins have molecular masses between 19-26 kD and consist of proteins with lengths of 156-166 or 172 amino acids. All IFN alpha subtypes possess a common conserved sequence region between amino acid positions 115-151 while the amino-terminal ends are variable. Many IFN alpha subtypes differ in their sequences only at one or two positions. Disulfide bonds are formed between cysteins at positions 1/98 and 29/138. The disulfide bond 29/138 is essential for biological activity while the 1/98 bond can be reduced without affecting biological activity. All IFN alpha forms contain a potential glycosylation site. Glycosylated IFN alpha forms are useful in the present invention.

Human IFN-γ is an ˜20 kDa factor produced by activated T, B and NK cells and is an anti-viral and anti-parasitic cytokine. The molecule contains two potential N-glycosylation sites. IFN-γ in synergy with other cytokines, such as TNF-α, inhibits proliferation of normal and transformed cells. Immunomodulatory effects of IFN-γ are exerted on a wide range of cell types expressing the high affinity receptors for IFN-γ. Glycosylation of IFN-γ does not affect its biological activity. Available under the name Actimmune (Genentech). Used for reducing the frequency and severity of serious infections associated with chronic granulomatous disease (Am J Ther. 1996 February; 3(2):109-114)

Antithrombin III (AT III) is a serine protease inhibitor that inhibits thrombin and factor Xa (Travis, Annu. Rev. Biochem. 52: 655, 1983). To a lesser extent, factor IXa, XIa, XIIa, tPA, urokinase, trypsin, plasmin and kallikrein are also inhibited (Menache, Semin. Hematol. 28:1, 1991; Menache, Transfusion 32:580, 1992; Lahiri, Arch. Biochem. Biophys. 175:737, 1976). Human AT III is synthesized in the liver as a single chain glycoprotein of 432 amino acids with a molecular weight (MW) of approximately 58,000 D. Its normal plasma concentration is within the range of 14-20 mg/dL (Rosenberg, Rev. Hematol. 2:351, 1986; Murano, Thromb. Res. 18:259, 1980). The protein bears three disulfide bridges (Cys 8-128, Cys 21-95, Cys 247-430) and four N-linked carbohydrate chains (Asn 96, -135, -155, -192) which account for 15% of the total mass (Franzen, J. Biol. Chem. 255:5090, 1980; Peterson, The Physiological Inhibitions of Blood Coagulation and Fibrinolysis, Elsevier/North-Holland Biomedical Press 1979, p. 43). AT III can be produced following classical human plasma fractionating techniques. Affinity chromatography (heparin-sepharose) using the high affinity of heparin for AT III followed by heat treatment for virus inactivation is used for the separation from plasma. More recent alternatives are available for the AT III production are recombinant production techniques that provide a safer access to this therapeutic Protein (Levi, Semin Thromb Hemost 27: 405, 2001). ATryn™ is a recombinant human AT III (rh AT III) produced by Genzyme Transgenics Corp. (GTC) in transgenic goats. The following AT III drugs are available on the European hospital market. (Source: IMS-ATC group 2001): Kybernin (Aventis Behring), AT III (Baxter, Grifols), Atenativ (Pharmacia), Aclotine (LFB), Grifols (Anbin).

Factor VII participates in the intrinsic blood coagulation cascade of proteinases and promoting hemostatsis by activating the extrinsic pathway of the coagulation cascade. F VII is converted to factor VIIa by factor Xa, factor XIIa, factor IXa, or thrombin by minor proteolysis. In the presence of tissue factor and calcium ions, factor VIIa then converts factor X to factor Xa by limited proteolysis. Factor VIIa will also convert factor IX to factor IXa in the presence of tissue factor and calcium. Factor VII is a vitamin K-dependent glycoprotein consisting of 406 amino acid residues (MW 50 kDalton). Factor VII is either produced by conventional extraction from donated human plasma or, more recently, using recombinant systems. Novo Nordisk uses Baby hamster kidney (BHK) cells for production of NovoSeven®. Expressed as the single-chain protein of 406 amino acids with a nominal molecular weight of 55 kDa (Thim, L. et al., Biochemistry 27:7785-7793 (1988)). The molecule bears four carbohydrate side chains. Two O-linked carbohydrate side chains at Ser 52, 60 and two N-linked carbohydrate side chains at Asn 145, 322 (Thim, L. et al., Biochemistry 27:7785-7793 (1988)).

Factor VIII participates in the intrinsic blood coagulation cascade of proteinases and serves as a cofactor in the reaction of factor IXa converting factor X to the active form, factor Xa, which ultimately leads to the formation of a fibrin clot. A lack or instability of factor VIII leads to haemophilia A, a common recessive x-linked coagulation disorder.

Factor VIII is either produced by conventional extraction from donated human plasma or, more recently, using recombinant systems. Bayer uses Baby hamster kidney (BHK) cells for production of Kogenate, whereas Baxter uses Chinese Hamster Ovary (CHO) cells for its product Recombinate. as the full single-chain protein of 2351 amino acids with a nominal molecular weight of 267 kDa (Toole et al., 1984, Nature 312: 342) or in different versions, where the full B-domain or parts of it are deleted in order to have a product that is more stable and gives a higher yield in production (Bhattacharyya et al., 2003, GRIPS 4/3: 2-8). A hesylated protein is expected to have a lower degree of immunogenicity and could thus reduce this complication.

Factor IX is a vitamin K-dependent plasma protein that participates in the intrinsic pathway of blood coagulation by converting factor X to its active form in the presence of Ca(2+) ions, phospholipids, and factor VIIIa. Factor IX is a glycoprotein with an approximate molecular mass of 55,000 Da consisting of 415 amino acids in a single chain (Yoshitake S. et al., Biochemistry 24:3736-3750 (1985)). Factor IX is either produced by conventional extraction from donated human plasma or, more recently, using recombinant systems. Wyeth uses Chinese hamster ovary (CHO) cells for production of BeneFIX®. It has a primary amino acid sequence that is identical to the Ala¹⁴⁸ allelic form of plasma-derived factor IX, and has structural and functional characteristics similar to those of endogenous factor IX. The protein bears eight carbohydrate side chains. Six O-linked carbohydrate side chains at Ser 53, 61 and at Threonine 159, 169, 172, 179 and two N-linked carbohydrate side chains at Asn 157, 167 (Yoshitake S. et al., Biochemistry 24:3736-3750 (1985); Balland A. et al., Eur J Biochem. 1988; 172(3):565-72).

Human granulocyte macrophage colony stimulating factor (hGM-CSF) is an early acting factor essential for regulation and differentiation of haematopoietic progenitor cells as well as for stimulating functional activation of mature cell populations. It has been cloned and expressed in yeast, bacteria, insect, plant and mammalian cells, resulting in a protein that varies in structure, composition, serum half-life and functions in vivo (Donahue, R. E.; Wang, E. A.; Kaufman, R. J.; Foutch, L.; Leary, A. C.; Witek-Giannetti, J. S.; Metzeger, M.; Hewick, R. M.; Steinbrink, D. R.; Shaw, G.; Kamen, R.; Clark, S. C. Effects of N-linked carbohydrates on the in vivo properties of human GM-CSF. Cold Spring Harbor Symp. Quant. Biol. 1986, 51, pp. 685-692). Natural and mammalian cell-derived hGM-CSF is a 127 amino acid protein and it contains both N- and O-glycans.). This lymphokine is of clinical interest due to its potential the treatment of myeloid leukemia and its ability to stimulate the granulocyte and macrophage production in patients suffering immunodeficiency or being suppressed by disease or radiation and/or chemotherapy (reviewed by Moonen, P.; Mermod, J. J.; Ernst, J. F.; Hirschi, M.; DeLamarter, J. F. Increased biological activity of deglycosylated recombinant human granulocyte-macrophage colony-stimulating factor produced by yeast or animal cells. Proc. Natl. Acad. Sci. US. 1987, 84, pp. 4428-4431). GM-CSF preparations are available under the names Leukine (Immunex) and Leucomax (Novartis). GM-CSF is used in myeloid reconstitution following bone marrow transplant, bone marrow transplant engraftment failure or delay, mobilization and following transplantation of autologous peripheral blood progenitor cells, and following induction chemotherapy in older adults with acute myelogenous leukemia.

Alpha1-Antitrypsin (A1AT, also referred to as alpha1-proteinase inhibitor) is a proteinase inhibitor that has been shown to inhibit virtually all mammalian serine proteinases (Travis Ann. Rev. Biochem. 52 (1983) p. 655) including neutrophil elastase, thrombin, factors Xa and XIa. A1AT is a single chain glycoprotein synthesized in the liver with 394 amino acids and a molecular weight of 53 kD. The plasma concentration is within a range of 1-1.3 g/l. The presence of only one cysteine in the whole protein does not allow the formation of intramolecular disulfide bridges. The molecule bears three carbohydrate side chains (Asn 46, 83, 247) (Mega J. Biol. Chem. 255 (1980) p. 4057; Mega J. Biol. Chem. 255 (1980) p. 4053; Carell FEBS Letters 135 (1981) p. 301; Hodges Biochemistry 21 (1982) p. 2805) that represent 12% of the molecular weight. The key function is the activity control of neutrophil elastase (Travis Ann. Rev. Biochem. 52 (1983) p. 655). An uncontrolled activity of elastase leads to an attack on epithelial tissues with the result of irreparable damage. During the inactivation process A1AT acts as a substrate for elastase binding to the active center of the protease which is subsequently inactivated by this complex formation. A deficiency of A1AT causes e.g. pulmonary emphysema which is in connected with a damage of the pulmonary epithelium. The distribution of the two types of carbohydrate side chains of A1AT to the three N-glycosylation sites of A1AT is different for each isotype of A1AT. The classical production of A1AT is conducted in human plasma fractionation using different affinity-chromatography steps. However a more recent way of producing A1AT is the use of recombinant techniques. PPL Therapeutics has developed a process that allows to recover recombinant human A1AT (rHA1AT) from the milk of transgenic sheep (Olman Biochem. Soc. Symp. 63 (1998) p. 141; Tebbutt Curr. Opin. Mol. Ther. 2 (2000) p. 199; Carver Cytotechnology 9 (1992) p. 77; Wright Biotechnology (NY) 9 (1991) p. 830).

The tissue type plasminogen activator (tPA) is a trypsine like serine protease important in clot lysis. In the presence of a fibrin clot, tPA converts plasminogen to plasmin, which degrades fibrin. TPA exhibits enhanced activity in the presence of fibrin and as a result, causes fibrin-specific plasminogen activation (M. W. Spellman, L. J. Basa, C. K. Leonard, J. A. Chakel, J. V. O'Connor, The Journal of Biological Chemistry 264 (1989) p. 14100). Plasmin solubilizes fibrin, yielding fibrin degradation products. Through a positive feedback mechanism, fibrin enhances its own degradation by stimulating tPA mediated plasminogen activation (R. J. Stewart et al., The Journal of Biological Chemistry 275 (2000) pp. 10112-10120). htPA is a physiological activator of fibrinolysis, which is present in different types of tissues. It is a glycoprotein with a molecular weight of approx. 68 kD. In native form tPA exists in a one-chain-form (single-chain tissue-type plasminogen activator, sctPA), which can be converted by cleavage of plasmin at the peptide bond Arg 275-Ile 276 to a two chain structure (two-chain tissue-type plasminogen activator, tctPA). For therapy of fibrinolysis it is produced recombinant as rtPA (recombinant tissue-type plasminogen activator). Different types of tPA exist showing structural differences in the carbohydrate structure. Type I tPA has N-linked oligosaccharides at amino acids Asn117, Asn184 and Asn448. Type II tPA is glycosylated at Asn117 and Asn448. Both types contain an O-linked fucose residue at Thr61 (K. Mori et al., The Journal of Biological Chemistry 270 (1995) pp. 3261-3267). Several results indicate that the in-vivo clearance of tPA is influenced by the carbohydrate structure, particularly by the high mannose oligosaccharide attached at site Asn117. Another proposed clearance mechanism involves the recognition of the O-linked fucose residue at Thr61 by a high affinity receptor on hepatocytes. TNK-tpA is on the market as Tenecteplase® (Boehringer Ingelheim) and can be administered as a single intravenous bolus, while tPA has to be administered as a bolus followed by an infusion.

Activated Protein C (APC) is a modulator of the coagulation and inflammation associated with severe sepsis. Activated Protein C is converted from its inactive precursor (protein C) by thrombin coupled to thrombomodulin. This complex cleaves off a short N-terminal activation peptide form the heavy chain of protein C, resulting in the activated protein C. Drotrecogin alpha (activated) is a recombinant human activated protein C (rhAPC) with an amino acid sequence identical to plasma derived activated protein C and with similar properties. Activated protein C is marketed by Eli Lilly as Xigris®. It is produced in a human cell line (HEK293), into which the protein C expression vectors were introduced. This particular cell line was used due to its ability to perform the correct series of complex post-translational modifications that are required for functional activity. Recombinant human activated protein C is a 2-chain glycoprotein containing 4 N-glycosylation sites and 12 disulfide bonds. The heavy chain contains 250 amino acids, of which seven residues are cysteines and it has three N-linked glycosylation sites (Asn-248, Asn-313 and Asn-329). The seven cysteine residues form three disulfide bonds within the heavy chain and one disulfide bond between the chains. The light chain contains one N-linked glycosylation site (Asn-97) and 17 cysteine residues, which form eight disulfide bonds within the light chain and one disulfide bond to the heavy chain. Activated protein C is a protease belonging to the serine protease family and plays a major role in the regulation of coagulation. Basis for the antithrombotic function of activated protein C is its ability to inhibit thrombin function. In addition, activated protein C is an important modulator of inflammation associated with severe sepsis. Due to its short physiological and pharmacokinetic half-life, activated protein C is continuously infused at a certain rate to maintain the desired plasma concentration in clinical use in sepsis therapy. Some effort is made to improve the pharmacokinetic profile of activated protein C. For example D. T. Berg et al., Proc. Natl. Acad. Sci. USA 100 (2003) pp. 4423-4428, describe an engineered variant of activated protein C with a prolonged plasma half-life.

As glycosylated protein, glycosylated forms of IFN beta such as natural human IFN beta or IFN beta 1a, natural or eucaryotic cell derived hGM-CSF containing both N- and O-glycans, recombinant human activated protein C (rhAPC) being a 2-chain glycoprotein containing 4 N-glycosylation sites, human tPA (htPA) or recombinant human tPA (rhtPA) such as type I tPA having N-linked oligosaccharides at amino acids Asn117, Asn184 and Asn448 or type II tPA being glycosylated at Asn117 and Asn448, plasma derived A1AT or recombinant human A1AT (pdA1AT or rhA1AT), recombinant human AT III (rhAT III), erythropoietin, factor VII, factor VIII and factor IX are preferred.

Glycosylated forms of EPO, IFN beta, AT III and GM-CSF are especially preferred.

The invention also relates to the conjugates obtainable by the method of the invention.

In the context of the present invention, the term “hydroxyalkyl starch” (HAS) refers to a starch derivative which has been substituted by at least one hydroxyalkyl group. A preferred hydroxyalkyl starch of the present invention has a constitution according to formula (I)

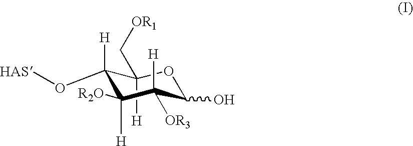

wherein the reducing end of the starch molecule is shown in the non-oxidized form and the terminal saccharide unit is shown in the acetal form which, depending on e.g. the solvent, may be in equilibrium with the aldehyde form.

The term hydroxyalkyl starch as used in the present invention is not limited to compounds where the terminal carbohydrate moiety comprises hydroxyalkyl groups R₁, R₂, and/or R₃ as depicted, for the sake of brevity, in formula (I), but also refers to compounds in which at least one hydroxy group present anywhere, either in the terminal carbohydrate moiety and/or in the remaining part of the starch molecule, HAS', is substituted by a hydroxyalkyl group R₁, R₂, or R₃.

Hydroxyalkyl starch comprising two or more different hydroxyalkyl groups are also possible.

The at least one hydroxyalkyl group comprised in HAS may contain two or more hydroxy groups. According to a preferred embodiment, the at least one hydroxyalkyl group comprised in HAS contains one hydroxy group.

The expression “hydroxyalkyl starch” also includes derivatives wherein the alkyl group is mono- or polysubstituted. In this context, it is preferred that the alkyl group is substituted with a halogen, especially fluorine, or with an aryl group. Furthermore, the terminal hydroxy group of a hydroxyalkyl group may be esterified or etherified.

Furthermore, instead of alkyl, also linear or branched substituted or unsubstituted alkene groups may be used.

Hydroxyalkyl starch is an ether derivative of starch. Besides of said ether derivatives, also other starch derivatives can be used in the context of the present invention. For example, derivatives are useful which comprise esterified hydroxy groups. These derivatives may be e.g. derivatives of unsubstituted mono- or dicarboxylic acids with 2-12 carbon atoms or of substituted derivatives thereof. Especially useful are derivatives of unsubstituted monocarboxylic acids with 2-6 carbon atoms, especially derivatives of acetic acid. In this context, acetyl starch, butyryl starch and propionyl starch are preferred.

Furthermore, derivatives of unsubstituted dicarboxylic acids with 2-6 carbon atoms are preferred.

In the case of derivatives of dicarboxylic acids, it is useful that the second carboxy group of the dicarboxylic acid is also esterified. Furthermore, derivatives of monoalkyl esters of dicarboxylic acids are also suitable in the context of the present invention.

For the substituted mono- or dicarboxylic acids, the substitute groups may be preferably the same as mentioned above for substituted alkyl residues.

Techniques for the esterification of starch are known in the art (see e.g. Klemm D. et al., Comprehensive Cellulose Chemistry Vol. 2, 1998, Whiley-VCH, Weinheim, N.Y., especially chapter 4.4, Esterification of Cellulose (ISBN 3-527-29489-9).

According to a preferred embodiment of the present invention, hydroxyalkyl starch according to formula (I) is employed.

In formula (I), the saccharide ring described explicitly and the residue denoted as HAS′ together represent the preferred hydroxyalkyl starch molecule. The other saccharide ring structures comprised in HAS′ may be the same as or different from the explicitly described saccharide ring.

As far as the residues R₁, R₂ and R₃ according to formula (I) are concerned there are no specific limitations. According to a preferred embodiment, R₁, R₂ and R₃ are independently hydrogen or a hydroxyalkyl group, a hydroxyaryl group, a hydroxyaralkyl group or a hydroxyalkaryl group having of from 2 to 10 carbon atoms in the respective alkyl residue or a group —(CH₂CH₂O)ₙ—H, wherein n is an integer, preferably 1, 2, 3, 4, 5 or 6. Hydrogen and hydroxyalkyl groups having of from 2 to 10 are preferred. More preferably, the hydroxyalkyl group has from 2 to 6 carbon atoms, more preferably from 2 to 4 carbon atoms, and even more preferably from 2 to 4 carbon atoms. In a preferred embodiment R₁, R₂ and R₃ according to formula (I) all are the same group —(CH₂CH₂O)ₙ—H, wherein n is an integer, preferably 1, 2 or 3. “Hydroxyalkyl starch” therefore preferably comprises hydroxyethyl starch, hydroxypropyl starch and hydroxybutyl starch, wherein hydroxyethyl starch and hydroxypropyl starch are particularly preferred and hydroxyethyl starch is most preferred.

The alkyl, aryl, aralkyl and/or alkaryl group may be linear or branched and optionally suitably substituted.

Therefore, the present invention also relates to a method as described above wherein R₁, R₂ and R₃ are independently hydrogen or a linear or branched hydroxyalkyl group with from 1 to 6 carbon atoms.

Thus, R₁, R₂ and R₃ preferably may be hydroxyhexyl, hydroxypentyl, hydroxybutyl, hydroxypropyl such as 2-hydroxypropyl, 3-hydroxypropyl, 2-hydroxyisopropyl, hydroxyethyl such as 2-hydroxyethyl, hydrogen and the 2-hydroxyethyl group being especially preferred.

Therefore, the present invention also relates to a method and a conjugate as described above wherein R₁, R₂ and R₃ are independently hydrogen or a 2-hydroxyethyl group, an embodiment wherein at least one residue R₁, R₂ and R₃ being 2-hydroxyethyl being especially preferred.

Hydroxyethyl starch (HES) is most preferred for all embodiments of the present invention.

Therefore, the present invention relates to the method and the conjugate as described above, wherein the polymer is hydroxyethyl starch and the polymer derivative is a hydroxyethyl starch derivative.

Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., 1987, Krankenhauspharmazie, 8(8), 271-278; and Weidler et al., 1991, Arzneim.-Forschung/Drug Res., 41, 494-498).

Amylopectin consists of glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. There are two possibilities of describing the substitution degree:
1. The degree of substitution can be described relatively to the portion of substituted glucose monomers with respect to all glucose moieties.
2. The degree of substitution can be described as the molar substitution, wherein the number of hydroxyethyl groups per glucose moiety are described.

In the context of the present invention, the degree of substitution, denoted as DS, relates to the molar substitution, as described above (see also Sommermeyer et al., 1987, Krankenhauspharmazie, 8(8), 271-278, as cited above, in particular p. 273).

HES solutions are present as polydisperse compositions, wherein each molecule differs from the other with respect to the polymerization degree, the number and pattern of branching sites, and the substitution pattern. HES is therefore a mixture of compounds with different molecular weight. Consequently, a particular HES solution is determined by average molecular weight with the help of statistical means. In this context, Mₙ is calculated as the arithmetic mean depending on the number of molecules. Alternatively, M_{w} (or MW), the weight mean, represents a unit which depends on the mass of the HES.

In the context of the present invention, hydroxyethyl starch may preferably have a mean molecular weight (weight mean) of from 1 to 300 kD. Hydroxyethyl starch can further exhibit a preferred molar degree of substitution of from 0.1 to 0.8 and a preferred ratio between C₂:C₆ substitution in the range of from 2 to 20 with respect to the hydroxyethyl groups.

The term “mean molecular weight” as used in the context of the present invention relates to the weight as determined according to the LALLS-(low angle laser light scattering)-GPC method as described in Sommermeyer, K., Cech, F., Schmidt, M., Weidler, B., 1987, Krankenhauspharmazie, 8(8), 271-278; and Weidler et al., 1991, Arzneim.-Forschung/Drug Res., 41, 494-498). For mean molecular weights of 10 kD and smaller, additionally, the calibration was carried out with a standard which had previously been qualified by LALLS-GPC.

According to a preferred embodiment of the present invention, the mean molecular weight of hydroxyethyl starch employed is from 1 to 300 kD, preferably from 2 to 200 kD, more preferably of from 3 to 130 kD, more preferably of from 4 to 100 kD, most preferably of from 4 to 90 kD.

An example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7.

An example for HES with a mean molecular weight of about 130 kD is Voluven® from Fresenius. Voluven® is an artificial colloid, employed, e.g., for volume replacement used in the therapeutic indication for therapy and prophylaxis of hypovolaemia. The characteristics of Voluven® are a mean molecular weight of 130,000+/−20,000 D, a molar substitution of 0.4 and a C2:C6 ratio of about 9:1.

Therefore, the present invention also relates to a method and to conjugates as described above wherein the hydroxyalkyl starch is hydroxyethyl starch having a mean molecular weight of from 4 to 100 kD, preferably 4 to 90 kD, more preferably 4 to 70 kD.

Preferred ranges of the mean molecular weight are, e.g., 4 to 90 kD or 10 to 90 kD or 12 to 90 kD or 18 to 90 kD or 50 to 90 kD or 70 to 90 kD or 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD.

According to particularly preferred embodiments of the present invention, the mean molecular weight of hydroxyethyl starch employed is in the range of from more than 4 kD and below 90 kD, such as about 10 kD, or in the range of from 9 to 10 kD or from 10 to 11 kD or from 9 to 11 kD, or about 12 kD, or in the range of from 11 to 12 kD or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

As far as the degree of substitution (DS) is concerned, DS is preferably at least 0.1, more preferably at least 0.2, and more preferably at least 0.4. Preferred ranges of DS are from 0.1 to 3, more preferably from 0.2 to 1.5, more preferably from 0.3 to 1.0, more preferably from 0.2 to 0.8, more preferably from 0.3 to 0.8 and even more preferably from 0.4 to 0.8, still more preferably from 0.1 to 0.7, more preferably from 0.2 to 0.7, more preferably from 0.3 to 0.7 and more preferably from 0.4 to 0.7. Particularly preferred values of DS are, e.g., 0.1, 0.2, 0.3, 0.4, 0.5, 0.6, 0.7 or 0.8, with 0.2, 0.3, 0.4, 0.5, 0.6, 0.7 or 0.8 being more preferred, 0.3, 0.4, 0.5, 0.6, 0.7 or 0.8 being even more preferred, 0.4, 0.5, 0.6, 0.7 or 0.8 being still more preferred and, e.g. 0.4 and 0.7 being particularly preferred.

As to the upper limit of the molar degree of substitution (DS), values of up to 3.0 such as 0.9, 1.0, 1.1, 1.2, 1.3, 1.4, 1.5, 1.6, 1.7, 1.8, 1.9 or 2.0 are also possible, values of below 2.0 being preferred, values of below 1.5 being more preferred, values of below 1.0 such as 0.7, 0.8 or 0.9 being still more preferred.

Therefore, preferred ranges of the molar degree of substitution are from 0.1 to 2 or from 0.1 to 1.5 or from 0.1 to 1.0 or from 0.1 to 0.9 or from 0.1 to 0.8. More preferred ranges of the molar degree of substitution are from 0.2 to 2 or from 0.2 to 1.5 or from 0.2 to 1.0 or from 0.2 to 0.9 or from 0.2 to 0.8. Still more preferred ranges of the molar degree of substitution are from 0.3 to 2 or from 0.3 to 1.5 or from 0.3 to 1.0 or from 0.3 to 0.9 or from 0.3 to 0.8. Even more preferred ranges of the molar degree of substitution are from 0.4 to 2 or from 0.4 to 1.5 or from 0.4 to 1.0 or from 0.4 to 0.9 or from 0.4 to 0.8.

In the context of the present invention, a given value of the molar degree of substitution such as 0.8 may be the exact value or may be understood as being in a range of from 0.75 to 0.84. Therefore, for example, a given value of 0.1 may be the exact value of 0.1 or be in the range of from 0.05 to 0.14, a given value of 0.4 may be the exact value of 0.4 or in the range of from 0.35 to 0.44, or a given value of 0.7 may be the exact value of 0.7 or be, in the range of from 0.65 to 0.74.

Particularly preferred combinations of molecular weight of the hydroxyalkyl starch, preferably hydroxyethyl starch, and its degree of substitution DS are, e.g., 10 kD and 0.4 or 10 kD and 0.7 or 12 kD and 0.4 or 12 kD and 0.7 or 18 kD and 0.4 or 18 kD and 0.7 or 30 kD and 0.4 or 30 kD and 0.7, or 50 kD and 0.4 or 50 kD and 0.7 or 100 kD and 0.7.

As far as the ratio of C₂:C₆ substitution is concerned, said substitution is preferably in the range of from 2 to 20, more preferably in the range of from 2 to 15 and even more preferably in the range of from 3 to 12.

According to a further embodiment of the present invention, also mixtures of hydroxyethyl starches may be employed having different mean molecular weights and/or different degrees of substitution and/or different ratios of C₂:C₆ substitution. Therefore, mixtures of hydroxyethyl starches may be employed having different mean molecular weights and different degrees of substitution and different ratios of C₂:C₆ substitution, or having different mean molecular weights and different degrees of substitution and the same or about the same ratio of C₂:C₆ substitution, or having different mean molecular weights and the same or about the same degree of substitution and different ratios of C₂:C₆ substitution, or having the same or about the same mean molecular weight and different degrees of substitution and different ratios of C₂:C₆ substitution, or having different mean molecular weights and the same or about the same degree of substitution and the same or about the same ratio of C₂:C₆ substitution, or having the same or about the same mean molecular weights and different degrees of substitution and the same or about the same ratio of C₂:C₆ substitution, or having the same or about the same mean molecular weight and the same or about the same degree of substitution and different ratios of C₂:C₆ substitution, or having about the same mean molecular weight and about the same degree of substitution and about the same ratio of C₂:C₆ substitution.

In different conjugates and/or different methods according to the present invention, different hydroxyalkyl starches, preferably different hydroxyethyl starches and/or different hydroxyalkyl starch mixtures, preferably different hydroxyethyl starch mixtures, may be employed.

In a still further preferred embodiment, the polymer or polymer derivative comprising functional group A is linked to a modified polyol introduced into the glycoprotein during step a) of the method for preparing a conjugate described above.

The oligosaccharide pattern of proteins produced in eukaryotic cells thus having been posttranslationally glycosylated, are not identical to the human derived proteins. Moreover, many glycosylated proteins do not have the desired number of terminal sialic acid residues masking a further carbohydrate moiety such as a galactose residue. Those further carbo-hydrate moieties such as a galactose residue, however, if not masked, are possibly responsible for disadvantages such as a shorter plasma half-life of the protein in possible uses of the protein as a medicament It was surprisingly found that by providing a protein conjugate formed by a hydroxyalkyl starch polymer, preferably a hydroxyethyl starch polymer, which is covalently linked, by the gentle method of the invention to a carbohydrate moiety of a carbohydrate side chain of the protein, either directly or via at least one linker compounds such as one or two linker compounds, it is possible to overcome at least the above mentioned disadvantage. Hence it is believed that by coupling a hydroxyalkyl starch polymer or derivative thereof, preferably a hydroxyethyl starch polymer or a derivative thereof, to at least one carbohydrate side chain of a glycosylated protein via a modified polyol, the lack of suitable terminal carbohydrate residues located at a carbohydrate side chain is compensated. According to another aspect of the invention, providing the respective conjugate with a hydroxyalkyl starch polymer or derivative thereof, preferably a hydroxyethyl starch polymer or a derivative thereof, coupled to the oxidized carbohydrate moiety as described above, does not only compensate the disadvantage but provides a protein conjugate having better characteristics in the desired field of use than the respective naturally occurring protein. Therefore, the respective conjugates according to the invention have a compensational and even a synergistic effect on the protein. It also possible that even proteins which are identical to human proteins or which are human proteins do not have the desired number of suitable masking terminal carbohydrate residues such as sialic acid residues at naturally occurring carbohydrate moieties. In such cases, providing the respective conjugate with a hydroxyalkyl starch polymer or derivative thereof, preferably a hydroxyethyl starch polymer or a derivative thereof, coupled to the enzymatically introduced modified polyol as described above, does not only overcome and compensate a disadvantage of an artificially produced protein, but improves the characteristics of a naturally occurring protein. As to the functional group of the hydroxyalkyl starch, preferably hydroxyethyl starch, or a derivative thereof, which is coupled to the introduced modified polyol, reference is made to the functional groups A as disclosed hereinunder. This general concept is not only applicable to glycosylated G-CSF, but principally to all glycosylated having said lack of terminal carbohydrate residues.

Among others, erythropoietin (EPO), IFN beta, GM-CSF, APC, tPA, A1AT, AT III, HCG, LH, FSH, an antibody fusion protein, a therapeutic antibody, an interleukin, especially interleukin 2 or 6, IFN-α, CSF, factor VII, factor VIII, and factor IX may be mentioned.

Therefore, the present invention also relates to the use of hydroxyalkyl starch, preferably hydroxyethyl starch, or a derivative thereof, for compensating the lack of terminal carbohydrate residues, preferably sialic acid residues, in naturally occurring or posttranslationally attached carbohydrate moieties of a protein, by covalently coupling the starch or derivative thereof to at least one modified polyol added enzymatically to a glycoprotein.

Accordingly, the present invention also relates to a method for compensating the lack of terminal carbohydrate residues, preferably sialic acid residues, in naturally occurring or posttranslationally attached carbohydrate moieties of a protein, by covalently coupling hydroxyalkyl starch, preferably hydroxyethyl starch, or a derivative thereof to at least one modified polyol added enzymatically to a glycoprotein.

Moreover, the present invention also relates to a conjugate formed by covalent linkage of a hydroxyalkyl starch, preferably hydroxyethyl starch, or a derivative thereof, to at least one modified polyol added enzymatically to a glycoprotein, said glycoprotein being either isolated from natural sources or produced by expression in eukaryotic cells, such as mammalian, insect or yeast cells, said modified polyol added enzymatically to a glycoprotein having at least one functional group Z, wherein the conjugate has in the desired field of use, preferably the use as medicament, the same or better characteristics than the respective unmodified protein.

According to one embodiment of the present invention, the functional group Z of the modified polyol is an aldehyde group, a hemiacetal group or a keto group. Therefore, the present invention relates to a method and conjugates as described above, wherein the functional group Z of the modified polyol is an aldehyde group, a hemiacetal group or a keto group.

In case the functional group Z of the modified polyol is an aldehyde group, a hemiacetal group or a keto group, functional group A of the polymer or the derivative thereof comprises an amino group according to the structure —NH—.

Therefore, the present invention also relates to a method and a conjugate as described above wherein the functional group A capable of being reacted with the optionally oxidized reducing end of the polymer, comprises an amino group according to structure —NH—.

According to one preferred embodiment of the present invention, this functional group A is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

Therefore, the present invention also relates to a method and a conjugate as described above wherein R′ is hydrogen or a methyl or a methoxy group.

According to another preferred embodiment of the present invention, the functional group A has the structure R′—NH—R″— where R″ preferably comprises the structure unit —NH— and/or the structure unit —(C=G)- where G is O or S, and/or the structure unit —SO₂—. According to more preferred embodiments, the functional group R″ is selected from the group consisting of

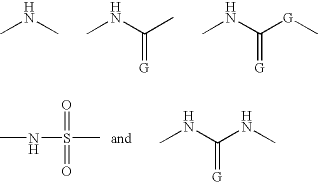

where, if G is present twice, it is independently O or S.

Therefore, preferred functional groups A comprising an amino group —NH₂, are, e.g.,

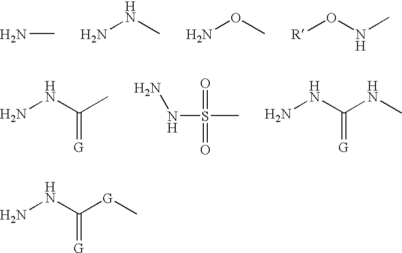

wherein G is O or S and, if present twice, independently O or S, and R′ is methyl.

Especially preferred functional groups A comprising an amino group are aminooxy groups

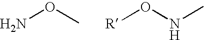

H₂N—O— being particularly preferred, and the hydrazido group

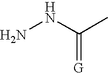

where G is preferably O.

Therefore, the present invention also relates to a method as described above, wherein the functional group Z of the modified polyol is an aldehyde group, a hemiacetal group or a keto group, and the functional group A is an aminooxy group or a hydrazido group. According to an especially preferred embodiment of the present invention, A is an aminooxy group.

Thus, the present invention also relates to a conjugate, as described above, wherein the functional group Z of the modified polyol is an aldehyde group or a keto group, and the functional group A is an aminooxy group or a hydrazido group. According to an especially preferred embodiment of the present invention, A is an aminooxy group.

When reacting the aminooxy group of the polymer or polymer derivative with the aldehyde group or keto group of the modified polyol, which has been transferred onto the glycoprotein during step a), an oxime linkage is formed.

Therefore, the present invention also relates to a conjugate as described above, wherein the covalent linkage between the modified polyol and the polymer or polymer derivative is an oxime linkage formed by the reaction of functional group Z of the modified polyol, said functional group Z being an aldehyde group, a hemiacetal group or a keto group, and functional group A of the polymer or polymer derivative, said functional group A being an aminooxy group.

When reacting the hydrazido group of the polymer or polymer derivative with the aldehyde group or keto group of the modified polyol, a hydrazone linkage is formed.

Therefore, the present invention also relates to a conjugate as described above, wherein the covalent linkage between the modified polyol and the polymer or polymer derivative is a hydrazone linkage formed by the reaction of functional group Z of the modified polyol, said functional group Z being an aldehyde group, a hemiacetal group or a keto group, and functional group A of the polymer or polymer derivative, said functional group A being a hydrazido group.

In order to introduce functional group A into the polymer, no specific restrictions exist given that a polymer derivative results comprising functional group A.

According to a preferred embodiment of the present invention, the functional group A is introduced into the polymer by reacting the polymer with an at least bifunctional compound, one functional group of which is capable of being reacted with at least one functional group of the polymer, and at least one other functional group of the at least bifunctional compound being functional group A or being capable of being chemically modified to give functional group A.

According to a still further preferred embodiment, the polymer is reacted with the at least bifunctional compound at its optionally oxidized reducing end.

In case the polymer is reacted with its non-oxidized reducing end, the polymer preferably has the constitution

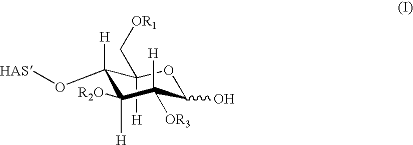

wherein in formula (I), the aldehyde form of the non-oxidized reducing end is included.

In case the polymer is reacted with its oxidized reducing end, the polymer preferably has the constitution according to formula (IIa)

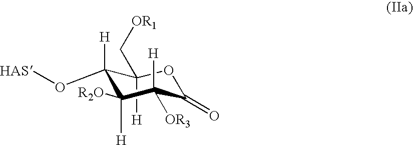

and/or according to formula (IIb)

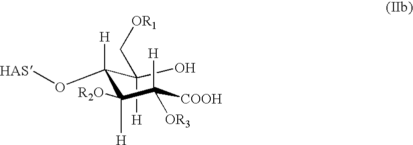

The oxidation of the reducing end of the polymer, preferably hydroxyethyl starch, may be carried out according to each method or combination of methods which result in compounds having the above-mentioned structures (IIa) and/or (IIb).

Although the oxidation may be carried out according to all suitable method or methods resulting in the oxidized reducing end of hydroxyalkyl starch, it is preferably carried out using an alkaline iodine solution as described, e.g., in DE 196 28 705 A1 the respective contents of which (example A, column 9, lines 6 to 24) is incorporated herein by reference.

As functional group of the at least bifunctional compound which is capable of being reacted with the optionally oxidized reducing end of the polymer, each functional group may be used which is capable of forming a chemical linkage with the optionally oxidized reducing end of the hydroxyalkyl starch.

According to a preferred embodiment of the present invention, this functional group comprises the chemical structure —NH—.

Therefore, the present invention also relates to a method and a conjugate as described above wherein the functional group of the at least bifunctional compound, said functional group being capable of being reacted with the optionally oxidized reducing end of the polymer, comprises the structure —NH—.

According to one preferred embodiment of the present invention, this functional group of the at least bifunctional compound is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

Therefore, the present invention also relates to a method and a conjugate as described above wherein R′ is hydrogen or a methyl or a methoxy group.

According to another preferred embodiment of the present invention, the functional group of the at least bifunctional compound has the structure R′—NH—R″—where R″ preferably comprises the structure unit —NH— and/or the structure unit —(C=G)- where G is O or S, and/or the structure unit —SO₂—. According to more preferred embodiments, the functional group R″ is selected from the group consisting of

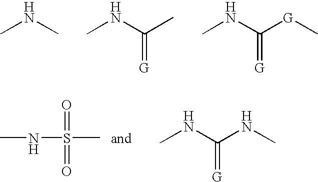

where, if G is present twice, it is independently O or S.

Therefore, the present invention also relates to a method and a conjugate as described above, wherein the functional group of the at least bifunctional compound, said functional group being capable of being reacted with the optionally oxidized reducing end of the polymer, is selected from the group consisting of

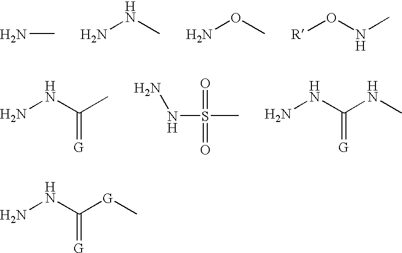

wherein G is O or S and, if present twice, independently O or S, and R′ is methyl.

According to an even more preferred embodiment of the present invention, the functional group of the at least bifunctional compound, said functional group being capable of being reacted with the optionally oxidized reducing end of the polymer and comprising an amino group, is an aminooxy groups

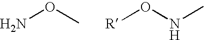

H₂N—O— being particularly preferred, or the hydrazido group

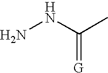

wherein G is preferably O.

Therefore, the present invention also relates to a method and a conjugate as described above, wherein the functional group Z of the modified polyol is an aldehyde group, a hemiacetal group or a keto group, and the functional group of the at least bifunctional compound, said functional group being capable of being reacted with the optionally oxidized reducing end of the polymer, is an aminooxy group or a hydrazido group, preferably an aminooxy group.

Thus, the present invention also relates to a conjugate, as described above, wherein the functional group Z of the modified polyol is an aldehyde group, a hemiacetal group or a keto group, and the functional group of the at least bifunctional compound, said functional group being capable of being reacted with the optionally oxidized reducing end of the polymer, is an aminooxy group or a hydrazido group, preferably an aminooxy group.

According to a still further preferred embodiment of the present invention, the at least bifunctional compound is reacted with the polymer at its non-oxidized reducing end.

According to yet another preferred embodiment of the present invention, the at least bifunctional compound which is reacted with the optionally oxidized reducing end of the polymer, comprises functional group A.

The at least bifunctional compound may be reacted with the polymer first to give a polymer derivative which is subsequently reacted with the protein via functional group A. It is also possible to react the at least bifunctional compound via functional group A with the modified polyol first to give a modified glycoprotein derivative which is subsequently reacted with the polymer via at least one functional group of the at least bifunctional compound residue comprised in the protein derivative.

According to a preferred embodiment of the present invention, the at least bifunctional compound is reacted with the polymer first.

Therefore, the present invention relates to a method and a conjugate as described above, said method further comprising reacting the polymer at its non-oxidized reducing end with an at least bifunctional linking compound comprising a functional group capable of reacting with the non-oxidized reducing end of the polymer and a group A, prior to the reaction of the polymer derivative comprising A and the modified polyol comprising Z.

The functional group of the at least bifunctional linking compound which is reacted with the polymer and the functional group A of the at least bifunctional linking compound which is reacted with functional group Z of the modified polyol may be separated by any suitable spacer. Among others, the spacer may be an optionally substituted, linear, branched and/or cyclic hydrocarbon residue. Generally, the hydrocarbon residue has up to 60, preferably up to 40, more preferably up to 20, more preferably up to 10, more preferably up to 6 and especially preferably up to 4 carbon atoms. If heteroatoms are present, the separating group comprises generally from 1 to 20, preferably from 1 to 8, more preferably 1 to 6, more preferably 1 to 4 and especially preferably from 1 to 2 heteroatoms. As heteroatom, O is preferred. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment of the present invention, the functional groups are separated by a linear hydrocarbon chain having 4 carbon atoms. According to another preferred embodiment of the present invention, the functional groups are separated by a linear hydrocarbon chain having 4 carbon atoms and at least one, preferably one heteroatom, particularly preferably an oxygen atom.

According to a further preferred embodiment, the at least bifunctional linking compound is a homobifunctional linking compound. Therefore, the present invention also relates to a method of producing a conjugate as described above, wherein the at least bifunctional linking compound is a homobifunctional compound.

Thus, with regard to the above mentioned preferred functional groups of the linking compound, said homobifunctional linking compound preferably comprises either two aminooxy groups H₂N—O— or two aminooxy groups R′—O—NH— or two hydrazido groups H₂N—NH—(C=G)-, the aminooxy groups H₂N—O— and the hydrazido groups H₂N—NH—(C═O)— being preferred, and the aminooxy groups H₂N—O— being especially preferred.

Among all conceivable homobifunctional compounds comprising two hydrazido groups H₂N—NH—(C═O)—, hydrazides are preferred where the two hydrazido groups are separated by a hydrocarbon residue having up to 60, preferably up to 40, more preferably up to 20, more preferably up to 10, more preferably up to 6 and especially preferably up to 4 carbon atoms. More preferably, the hydrocarbon residue has 1 to 4 carbon atoms such as 1, 2, 3, or 4 carbon atoms. Most preferably, the hydrocarbon residue has 4 carbon atoms. Therefore, a homobifunctional compound according to formula

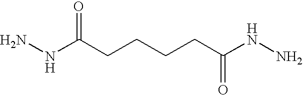

is preferred.

In the above-described embodiment where an aldehyde group or a keto group of the modified polyol is reacted with a compound comprising two hydrazido groups H₂N—NH—(C═O)—, particularly preferred hydroxyethyl starches are, e.g., hydroxyethyl starches having a mean molecular weight of about 10 kD and a DS of about 0.4. Also possible are, e.g., hydroxyethyl starch having a mean molecular weight of about 10 kD and a DS of about 0.7 or hydroxyethyl starch having a mean molecular weight of about 18 kD and a DS of about 0.4 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 12 kD and a DS of about 0.4 or hydroxyethyl starch having a mean molecular weight of about 12 kD and a DS of about 0.7 or hydroxyethyl starch having a mean molecular weight of about 18 kD and a DS of about 0.7 or hydroxyethyl starch having a mean molecular weight of about 30 kD and a DS of about 0.4 or hydroxyethyl starch having a mean molecular weight of about 30 kD and a DS of about 0.7 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

According to an even more preferred embodiment of the present invention, the bifunctional linking compound is carbohydrazide

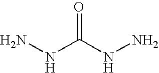

In the above-described embodiment where an aldehyde group or a keto group of the protein is reacted with carbohydrazide, particularly preferred hydroxyethyl starches are, e.g., hydroxyethyl starches having a mean molecular weight of about 10 kD and a DS of about 0.4. Also possible are, e.g., hydroxyethyl starch having a mean molecular weight of about 10 kD and a DS of about 0.7 or hydroxyethyl starch having a mean molecular weight of about 18 kD and a DS of about 0.4 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 12 kD and a DS of about 0.4 or hydroxyethyl starch having a mean molecular weight of about 12 kD and a DS of about 0.7 or hydroxyethyl starch having a mean molecular weight of about 18 kD and a DS of about 0.7 or hydroxyethyl starch having a mean molecular weight of about 30 kD and a DS of about 0.4 or hydroxyethyl starch having a mean molecular weight of about 30 kD and a DS of about 0.7 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

As described above, the present invention also relates to a method and a conjugate as described above, wherein the at least bifunctional linking compound is a homobifunctional compound and comprises two aminooxy groups. Hence, the present invention also relates to a method and a conjugate as described above, wherein the at least bifunctional linking compound is a homobifunctional compound and comprises two aminooxy groups H₂N—O—.

As described above, the polymer is preferably reacted at its reducing end which is not oxidized prior to the reaction with the bifunctional linking compound. Therefore, reacting the preferred homobifunctional compound comprising two aminooxy groups H₂N—O— with the polymer results in a polymer derivative comprising an oxime linkage.

Therefore, since functional group Z of the modified polyol is an aldehyde, a hemiacetal or a keto group which is preferably reacted with an aminooxy group of the polymer derivative, the present invention also relates to a conjugate as described above, said conjugate comprising the polymer and the glycoprotein, wherein the polymer and the modified polyol are each covalently linked to a linking compound by an oxime or a cyclic aminal linkage.

Among all conceivable homobifunctional compounds comprising two aminooxy groups H₂N—O—, bifunctional compounds are preferred where the two aminooxy groups are separated by a hydrocarbon residue having from 1 to 60, preferably from 1 to 40, more preferably from 1 to 20, more preferably from 1 to 10, more preferably from 1 to 6 and especially preferably 1 to 4 carbon atoms. More preferably, the hydrocarbon residue has 1 to 4 carbon atoms such as 1, 2, 3, or 4 carbon atoms. Most preferably, the hydrocarbon residue has 4 carbon atoms. Even more preferably, the hydrocarbon residue has at least one heteroatom, more preferably one heteroatom, and most preferably one oxygen atom. The compound O-[2-(2-aminooxy-ethoxy)-ethyl]hydroxylamine according to formula

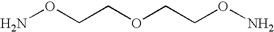

is especially preferred.

Therefore, the present invention relates to a conjugate as described above, said conjugate having a constitution according to formula

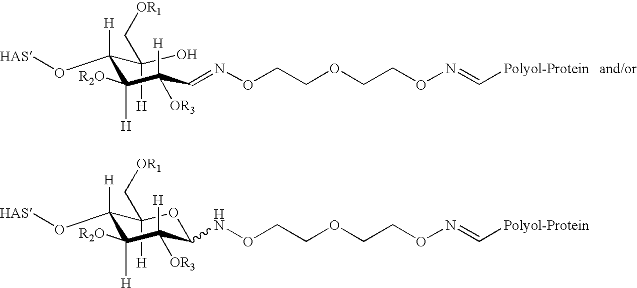

HAS′ preferably being HES′. Particularly preferred hydroxyethyl starches are, e.g., hydroxethyl starches having a mean molecular weight of about 10 kD and a DS of about 0.4 or hydroxethyl starch having a mean molecular weight of about 10 kD and a DS of about 0.7 or hydroxethyl starch having a mean molecular weight of about 12 kD and a DS of about 0.4 or hydroxethyl starch having a mean molecular weight of about 12 kD and a DS of about 0.7 or hydroxethyl starch having a mean molecular weight of about 18 kD and a DS of about 0.4 or hydroxethyl starch having a mean molecular weight of about 18 kD and a DS of about 0.7 or hydroxethyl starch having a mean molecular weight of about 30 kD and a DS of about 0.4 or hydroxyethyl starch having a mean molecular weight of about 30 kD and a DS of about 0.7 or hydroxyethyl starch having a mean molecular weight of about 50 kD and a DS of about 0.4 or hydroxethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

In the above-described embodiment where an aldehyde group or a keto group of the modified polyol is reacted with a hydroxylamino group of the polymer or polymer derivative, particularly preferred hydroxyethyl starches are, e.g., hydroxyethyl starches having a mean molecular weight of about 10 kD and a DS of about 0.4 and hydroxyethyl starch having a mean molecular weight of about 10 kD and a DS of about 0.7 and hydroxyethyl starch having a mean molecular weight of about 18 kD and a DS of about 0.4 and hydroxyethyl starch having a mean molecular weight of about 50 kD and a DS of about 0.4 or hydroxyethyl starch having a mean molecular weight of about 50 kD and a DS of about 0.7. Also possible are, e.g., hydroxyethyl starch having a mean molecular weight of about 12 kD and a DS of about 0.4 or hydroxyethyl starch having a mean molecular weight of about 12 kD and a DS of about 0.7 or hydroxyethyl starch having a mean molecular weight of about 18 kD and a DS of about 0.7 or hydroxyethyl starch having a mean molecular weight of about 30 kD and a DS of about 0.4 or hydroxyethyl starch having a mean molecular weight of about 30 kD and a DS of about 0.7 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

As glycoproteins, erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX are preferred.

The reaction of the polymer at its non-oxidized reducing end with the linking compound, especially in the case said linking compound is a homobifunctional linking compound comprising two aminooxy groups H₂N—O—, is preferably carried out in an aqueous system.

The term “aqueous system” as used in the context of the present invention refers to a solvent or a mixture of solvents comprising water in the range of from at least 10% per weight, preferably at least 50% per weight, more preferably at least 80% per weight, even more preferably at least 90% per weight or up to 100% per weight, based on the weight of the solvents involved. The preferred reaction medium is water.

According to another embodiment, at least one other solvent may be used in which HAS, preferably HES is soluble. Examples of these solvents are, e.g., DMF, dimethylacetamide or DMSO.

As far as the temperatures which are applied during the reaction are concerned, no specific limitations exist given that the reaction results in the desired polymer derivative.

In case the polymer is reacted with the homobifunctional linking compound comprising two aminooxy groups H₂N—O—, preferably O-[2-(2-aminooxy-ethoxy)-ethyl]hydroxyl amine, the temperature is preferably in the range of from 0 to 45° C., more preferably in the range of from 4 to 37° C. and especially preferably in the range of from 15 to 25° C.

The reaction time for the reaction of the polymer with the homobifunctional linking compound comprising two aminooxy groups H₂N—O—, preferably O-[2-(2-aminooxy-ethoxy)-ethyl]hydroxylamine, may be adapted to the specific needs and is generally in the range of from 1 h to 7 d, preferably in the range of from 1 h to 3 d and more preferably of from 2 h to 48 h.

The pH value for the reaction of the polymer with the homobifunctional linking compound comprising two aminooxy groups H₂N—O—, preferably O-[2-(2-aminooxy-ethoxy)-ethyl]hydroxylamine, may be adapted to the specific needs such as the chemical nature of the reactants. The pH value is preferably in the range of from 4.5 to 6.5.

Specific examples of above mentioned reaction conditions are, e.g., a reaction temperature of about 25° C. and a pH of about 5.5.

The suitable pH value of the reaction mixture may be adjusted by adding at least one suitable buffer. Among the preferred buffers, sodium acetate buffer, phosphate or borate buffers may be mentioned.

Once the polymer derivative comprising the polymer and the bifunctional linking compound linked thereto is formed, it may be isolated from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures, such as, for example acetone/ethanol mixtures in suitable volume/volume ratios, such as 1/1 v/v or isopropanol at suitable temperatures such as from −20° C. to 50° C. or from 0° C. to 25° C. According to a particularly preferred embodiment of the present invention where an aqueous medium, preferably water is used as solvent, the reaction mixture is contacted with a mixture of 2-propanol at a temperature, preferably in the range of from −20 to +50° C. and especially preferably in the range of from 0 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

The thus isolated polymer derivative is then further reacted, via functional group A, with the functional group Z of the modified polyol, Z being an aldehyde group, a hemiacetal or a keto group. In the especially preferred case that A is an aminooxy group H₂N—O— to give an oxime linkage between polymer derivative and modified polyol, the reaction is preferably carried out in an aqueous medium, preferably water, at a preferred temperature in the range of from 0 to 40° C., more preferably from 1 to 25° C. and especially preferably from 15 to 25° C. or alternatively from 1 to 15° C. The pH value of the reaction medium is preferably in the range of from 4 to 10, more preferably in the range of from 5 to 9 and especially preferably in the range of from 5 to 7. The reaction time is preferably in the range of from 1 to 72 h, more preferably in the range of from 1 to 48 h and especially preferably in the range of from 4 to 24 h.

The conjugate may be subjected to a further treatment such as an after-treatment like dialysis, centrifugal filtration or pressure filtration, ion exchange chromatography, reversed phase chromatography, HPLC, MPLC, gel filtration and/or lyophilisation.

The present invention relates to a method as described above, wherein the functional group Z of the polyol and the functional group A linked by a chemical residue according to formula (I)

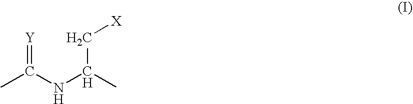

wherein Y is a heteroatom, selected from the group consisting of O and S, said method comprising reacting group Z, being a thioester group —(C═Y)—S—R′ with group Z, being an alpha-X beta-amino group

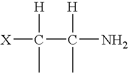

wherein R′ is selected from the group consisting of hydrogen, an optionally suitably substituted, linear, cyclic and/or branched alkyl, aryl, heteroaryl, aralkyl, and heteroaralkyl group, preferably benzyl.

Therefore, the term “alpha-X beta-amino group” as used in the context of the present invention refers to an ethylene group in which X is bonded to a carbon atom and a primary amino group is bonded to the neighbouring carbon atom.

In the chemical moiety according to formula (I) above, the group —(C═Y) is derived from the thioester group —(C═Y)—S—R′ and the group HN—CH—CH₂—X is derived from the alpha-X beta amino group.

The invention also relates to the embodiments as described on pages 20 to 34, wherein the position of groups Z and A is reversed, in particular, wherein the functional group introduced into the glycoprotein during step a) of the method of the invention is a group containing an amino group and wherein the reactive group Z of the polymer or polymer derivative is an aldehyde group, a hemiacetal group or a keto group. It is particularly preferred that the group Z is selected from a hydroxylamine, a hydrazine, a hydrazid or a hydrazide derivative as described above. In those cases, wherein Z is a hydroxylamino group or a hydrazido group, the polymer, e.g. the HAS, to be used in step b needs not to be modified in order to be able to form a covalent linkage with the glycoprotein obtained from step a).

According to another embodiment of the present invention, the functional group Z of the modified polyol is an amino group and the glycoprotein is preferably selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX.

Therefore, the present invention relates to a method and a conjugate as described above, wherein the functional group Z of the protein is an amino group and the protein is selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX.

According to an especially preferred embodiment of the present invention, the functional group A to be reacted with the functional group Z being an amino group is a reactive carboxy group. Therefore, the present invention also relates to a method and a conjugate as described above, wherein the functional group Z is an amino group and the functional group A of the polymer or the polymer derivative is a reactive carboxy group.

In the above-described embodiment where an amino group of the modified polyol is reacted with a reactive carboxy group of the polymer or polymer derivative, particularly preferred hydroxyethyl starches are, e.g., hydroxyethyl starches having a mean molecular weight of about 10 kD and a DS of about 0.4. Also possible are hydroxyethyl starch having a mean molecular weight of about 10 kD and a DS of about 0.7 and hydroxyethyl starch having a mean molecular weight of about 18 kD and a DS of about 0.4 and hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 12 kD and a DS of about 0.4 or hydroxyethyl starch having a mean molecular weight of about 12 kD and a DS of about 0.7 or hydroxyethyl starch having a mean molecular weight of about 18 kD and a DS of about 0.7 or hydroxyethyl starch having a mean molecular weight of about 30 kD and a DS of about 0.4 or hydroxyethyl starch having a mean molecular weight of about 30 kD and a DS of about 0.7 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

The reaction of the reactive polymer with the modified polyol, attached to the glycoprotein during step a), may be carried out by combining the reaction mixture of the preparation of the reactive polymer, i.e. without isolation of the reactive polymer, comprising at least 10, more preferably at least 30 and still more preferably at least 50 percent by weight reactive polymer, with an aqueous solution of the protein. Preferred aqueous solutions of the protein comprises of from 0.05 to 10, more preferably of from 0.5 to 5 and especially preferably of from 0.5 to 2 percent by weight protein at a preferred pH of from 5.0 to 9.0, more preferably of from 6.0 to 9.0 and especially preferably of from 7.5 to 8.5.

According to the present invention, it is also possible to purify the reactive polymer by at least one, preferably multiple precipitation with at least one suitable precipitation agent such as anhydrous ethanol, isopropanol and/or acetone to give a solid comprising at least 10, more preferably at least 30 and still more preferably at least 50 percent by weight reactive polymer.

The purified reactive polymer may be added to the aqueous solution of the modified glycoprotein. It is also possible to add a solution of the purified reactive polymer to the aqueous solution of the modified glycoprotein.

According to a preferred embodiment of the present invention, the reaction of the reactive polymer with the protein to give an amide linkage is carried out at a temperature of from 0 to 40° C., more preferably from 1 to 25° C. and especially preferably from 15 to 25° C. or alternatively from 1 to 15° C., and a preferred pH of from 7.0 to 9.0, preferably of from 7.5 to 9.0 and especially preferably of from 7.5 to 8.5, at a preferred reaction time of from 0.1 to 12 h, more preferably of from 0.5 to 5 h, more preferably of from 0.5 to 3 h, still more preferably of from 0.5 to 2 h and especially preferably of from 0.5 to 1 h, the molar ratio of reactive polymer ester:protein being preferably of from 1:1 to 70:1, more preferably of from 5:1 to 50:1 and especially preferably of from 10:1 to 50:1.

According to a further especially preferred embodiment of the present invention, the functional group A to be reacted with the functional group Z being an amino group is an aldehyde group, a keto group or a hemiacetal group. Therefore, the present invention also relates to a method and a conjugate as described above, wherein the functional group Z is an amino group and the functional group A of the polymer or the derivative thereof is an aldehyde group, a keto group or a hemiacetal group. Preferably, the glycoprotein is selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX.

According to a particularly preferred embodiment, functional group Z and functional group A are reacted via a reductive amination reaction.

According to this preferred embodiment, it is preferred to react the polymer at its optionally oxidized reducing end with an at least bifunctional compound comprising an amino group M and a functional group Q, wherein said amino group M is reacted with the optionally oxidized reducing end of the polymer and wherein the functional group Q is chemically modified to give an polymer comprising functional group A derivative which is reacted with an amino group Z by reductive amination.

The term “the polymer is reacted via the reducing end” or “the polymer is reacted via the oxidized reducing end” as used in the context of the present invention may relate to a process according to which the hydroxyalkyl starch is reacted predominantly via its (selectively oxidized) reducing end. The polymer is hydroxyalkyl starch, in particular hydroxyethyl starch.

This term “predominantly via its (selectively oxidized) reducing end” relates to processes according to which statistically more than 50%, preferably at least 55%, more preferably at least 60%, more preferably at least 65%, more preferably at least 70%, more preferably at least 75%, more preferably at least 80%, more preferably at least 85%, more preferably at least 90%, and still more preferably at least 95% such as 95%, 96%, 97%, 98%, or 99% of the polymer molecules employed for a given reaction are reacted via at least one (selectively oxidized) reducing end per polymer molecule, wherein a given polymer molecule which is reacted via at least one reducing end can be reacted in the same given reaction via at least one further suitable functional group which is comprised in said polymer molecule and which is not a reducing end. If one or more polymer molecule(s) is (are) reacted via at least one reducing and simultaneously via at least one further suitable functional group which is comprised in this (these) polymer molecule(s) and which is not a reducing end, statistically preferably more than 50%, preferably at least 55%, more preferably at least 60%, more preferably at least 65%, more preferably at least 70%, more preferably at least 75%, more preferably at least 80%, more preferably at least 85%, more preferably at least 90%, and still more preferably at least 95% such as 95%, 96%, 97%, 98%, or 99% of all reacted functional groups of these polymer molecules, said functional groups including the reducing ends, are reducing ends.

The term “reducing end” as used in the context of the present invention relates to the terminal aldehyde group of a polymer molecule which may be present as aldehyde group and/or as corresponding acetal form. In case the reducing end is oxidized, the aldehyde or acetal group is in the form of a carboxy group and/or of the corresponding lactone.

As to functional group Q, the following functional groups are to be mentioned, among others:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2 amino-thioalcohols;
1,2-aminoalcohols;
the amino group —NH₂ or derivatives of the amino groups comprising the structure unit —NH— such as aminoalkyl groups, aminoaryl group, aminoaralkyl groups, or alkarlyaminogroups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxalalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxylamines having imid structure such as N-hydroxysuccinimide or having a structure unit O—N where N is part of a heteroaryl compound or, with G=0 and Q absent, such as aryloxy compounds with a substituted aryl residue such as pentafluorophenyl, paranitrophenyl or trichlorophenyl;
wherein Q is absent or NH or a heteroatom such as S or O;
—NH—NH₂, or —NH—NH—;
—NO₂;
the nitril group;
carbonyl groups such as the aldehyde group or the keto group;
the carboxy group;
the —N═C═O group or the —N═C═S group;
vinyl halide groups such as the vinyl iodide or the vinyl bromide group or triflate;
—C═C—H;
—(C═NH₂Cl)-OAlkyl
groups —(C═O)—CH₂-Hal wherein Hal is Cl, Br, or I;
—CH═CH—SO₂—;
a disulfide group comprising the structure —S—S—;
the group

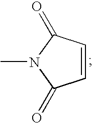

the group

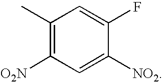

According to a preferred embodiment of the present invention, the term “functional group Q” relates to a functional group Q which comprises the chemical structure —NH—, e.g. —NH₂ or a derivative of the amino group comprising the structure unit —NH— such as aminoalkyl groups, aminoaryl group, aminoaralkyl groups, or alkarlyaminogroups.

According to one preferred embodiment of the present invention, the functional group M is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

According to another embodiment of the present invention, the functional group M has the structure R′—NH—R″—where R″ preferably comprises the structure unit —NH— and/or the structure unit —(C=G)- where G is O or S, and/or the structure unit —SO₂—. Specific examples for the functional group R″ are

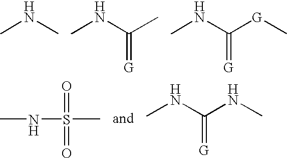

where, if G is present twice, it is independently O or S.

Therefore, the present invention also relates to a method and a conjugate as mentioned above wherein the functional group M is selected from the group consisting of

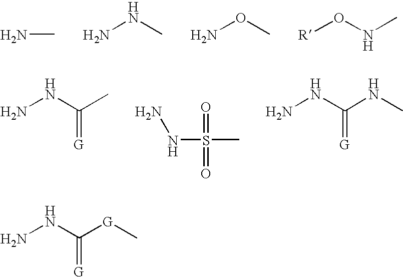

wherein G is O or S and, if present twice, independently O or S, and R′ is methyl.

According to a particularly preferred embodiment of the present invention, the functional group M is an amino group —NH₂.

According to a first alternative, the functional group M being an amino group NH₂ is reacted with the oxidized reducing end of the polymer resulting in an amido group linking the polymer and the compound comprising M and Q.

According to a second alternative, the functional group M being an amino group NH₂ is reacted with the non-oxidized reducing end of the polymer via reductive amination resulting in an imino group which is subsequently preferably hydrogenated to give an amino group, the imino group and the amino group, respectively, linking the polymer and the compound comprising M and Q. In this case, it is possible that the functional group Q is an amino group. In case that the resulting polymer derivative shall be subjected to a subsequent reaction with an at least bifunctional compound via a carboxy group or a reactive carboxy group, as described hereinunder, or another group of an at least bifunctional compound which is to be reacted with an amino group, it is preferred that the compound comprising M and Q is a primary amine which contains—as functional group—only one amino group. In this specific case, although the compound contains only one functional group, it is regarded as bifunctional compound comprising M and Q wherein M is the amino group contained in the compound subjected to the reductive amination with the reducing end of the polymer, and wherein Q is the secondary amino group resulting from the reductive amination and subsequent hydrogenation.

According to a third alternative, the non-oxidized reducing end of the polymer is reacted with ammonia via reductive amination resulting in a terminal imino group of the polymer which is subsequently preferably hydrogenated to give a terminal amino group of the polymer and thus a terminal primary amino group. In this specific case, ammonia is regarded as bifunctional compound comprising M and Q wherein M is NH₂ comprised in the ammonia employed, and wherein Q is the primary amino group resulting from reductive amination and subsequent hydrogenation.

The term “amino group Q” relates to a functional group Q which comprises the chemical structure —NH—, e.g. —NH₂ or a derivative of the amino group comprising the structure unit —NH— such as aminoalkyl groups, aminoaryl group, aminoaralkyl groups, or alkarlyaminogroups.

According to a preferred embodiment of the present invention, the functional group Q is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

According to another embodiment of the present invention, the functional group Q has the structure R′—NH—R″—where R″ preferably comprises the structure unit —NH— and/or the structure unit —(C=G)- where G is O or S, and/or the structure unit —SO₂—. According to more preferred embodiments, the functional group R″ is selected from the group consisting of

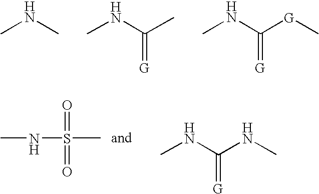

where, if G is present twice, it is independently O or S.

Therefore, the present invention also relates to a method and a conjugate as mentioned above wherein the functional group Q is selected from the group consisting of

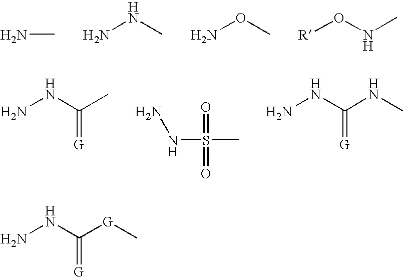

wherein G is O or S and, if present twice, independently O or S, and R′ is methyl.

According to a particularly preferred embodiment of the present invention, the functional group Q is an amino group —NH₂.

According to a still further preferred embodiment of the present invention, both M and Q comprise an amino group —NH—. According to a particularly preferred embodiment, both M and Q are an amino group —NH₂.

According to a preferred embodiment of the present invention, the compound comprising M and Q is a homobifunctional compound, more preferably a homobifunctional compound comprising, as functional groups M and Q, most preferably the amino group —NH₂, or according to other embodiments, the hydroxylamino group —O—NH₂ or the group

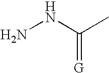

with G preferably being O. Specific examples for these compounds comprising M and Q are

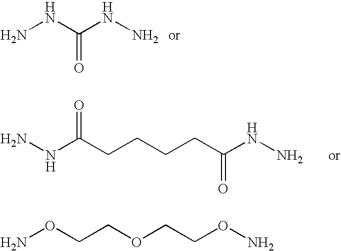

In case both M and Q are an amino group —NH₂, M and Q may be separated by any suitable spacer. Among others, the spacer may be an optionally substituted, linear, branched and/or cyclic hydrocarbon residue. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 2 to 10, more preferably from 2 to 6, and especially preferably from 2 to 4 carbon atoms.

Therefore, the present invention also relates to a method and a conjugate as described above, wherein the polymer is reacted with 1,4-diaminobutane, 1,3-diaminopropane or 1,2-diaminoethane to give a polymer derivative.

The reaction of the at least bifunctional compound comprising M and Q with the polymer is preferably carried out at a temperature of from 0 to 100° C., more preferably of from 4 to 80° C. and especially preferably of from 20 to 80° C.; the reaction time preferably ranges of from 4 h to 7 d, more preferably of from 10 h to 5 d and especially preferably of from 17 to 4 h. The molar ratio of at least bifunctional compound: polymer is preferably in the range of from 10 to 200, especially from 50 to 100.

As solvent for the reaction of the at least bifunctional compound with the polymer, at least one aprotic solvent, particularly preferably an anhydrous aprotic solvent having a water content of not more than 0.5 percent by weight, preferably of not more than 0.1 percent by weight is preferred. Suitable solvents are, among others, dimethyl sulfoxide (DMSO), N-methylpyrrolidone, dimethyl acetamide (DMA), dimethyl formamide (DMF) and mixtures of two or more thereof.

As solvent for the reaction of the at least bifunctional compound with the polymer, also an aqueous medium may be used.

According to a preferred embodiment, the polymer derivative comprising the polymer and the at least bifunctional compound is chemically modified at the free functional group Q to give a polymer derivative comprising an aldehyde group or keto group or hemiacetal group A. According to this embodiment, it is preferred to react the polymer derivative with at least one at least bifunctional compound which comprises a functional group capable of being reacted with the functional group Q and an aldehyde group or keto group or hemiacetal group.

As at least bifunctional compound, each compound is suitable which has an aldehyde group or keto group or hemiacetal group and at least one functional group which is capable of forming a linkage with the functional group Q of the polymer derivative. The at least one functional group is selected from the same pool of functional groups as Q and is chosen to be able to be reacted with Q. In the preferred case that Q is an amino group —NH₂, or a derivative of the amino group comprising the structure unit —NH— such as aminoalkyl groups, aminoaryl group, aminoaralkyl groups, or alkarylamino groups it is preferred to employ a compound having, apart from the aldehyde group or keto group or hemiacetal group, at least one carboxy group or at least one reactive carboxy group, preferably one carboxy group or one reactive carboxy group. The aldehyde group or keto group or hemiacetal group and the carboxy group or the reactive carboxy group may be separated by any suitable spacer. Among others, the spacer may be an optionally substituted, linear, branched and/or cyclic hydrocarbon residue. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group.

According to a preferred embodiment, the hydrocarbon residue is an alkyl group having 2 to 6 and preferably 2 to 4 carbon atoms. It is also possible that no carbon atom is present between the aldehyde or keto group and the carboxy group. Alternatively, the hydrocarbon residue can be a substituted or unsubstituted cyclic hydrocarbon group having 3 to 11 carbon atoms, preferably, 3 to 6 or 3 to 5 carbon atoms. When the cyclic hydrocarbon group is substituted, the substituent can be selected from the group consisting of substituted or unsubstituted amino or alkoxy groups. If present, the number of substituents is preferably 1 to 3. Further, the alkyl and/or cyclic hydrocarbon group can contain one or more heteroatoms, such as O or S, in particular O. In this case, preferably 1 to 3, in particular 1 or 2 heteroatoms are present. Preferred compounds in this context are selected from the following group of compounds.

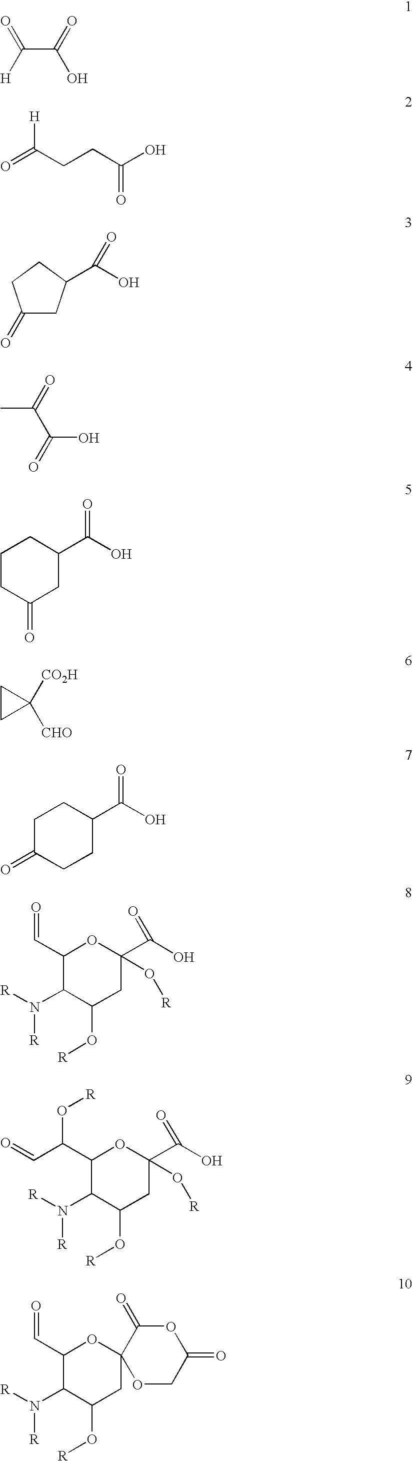

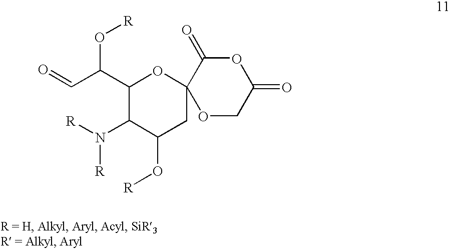

According to an even more preferred embodiment, the hydrocarbon residue is an aryl residue having 5 to 7 and preferably 6 carbon atoms. Most preferably, the hydrocarbon residue is the benzene residue. According to this preferred embodiment, the carboxy group and the aldehyde group may be located at the benzene ring in 1,4-position, 1,3-position or 1,2-position, the 1,4-position being preferred.

As reactive carboxy group, a reactive ester, isothiocyanates or isocyanate may be mentioned. Preferred reactive esters are derived from N-hydroxy succinimides such as N-hydroxy succinimide or Sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, or hydroxyazoles such as hydroxy benzotriazole. Especially preferred are N-hydroxy succinimides, with N-hydroxy succinimide and Sulfo-N-hydroxy succinimide being especially preferred. All alcohols may be employed alone or as suitable combination of two or more thereof. As reactive esters, pentafluorophenyl ester and N-hydroxy succinimide ester are especially preferred.

According to a specific embodiment, the functional group which is capable of forming a chemical linkage with the functional group Q, Q preferably being NH₂ or a derivative of the amino group comprising the structure unit —NH— such as aminoalkyl groups, aminoaryl group, aminoaralkyl groups, or alkarylamino groups, in particular being NH₂, is a reactive carboxy group.

In this case, the functional group which is capable of forming a chemical linkage with the functional group Q and which is a carboxy group, is suitably reacted to obtain a reactive carboxy group as described hereinabove. Therefore, it is preferred to subject the at least one at least bifunctional compound which comprises a carboxy group and an aldehyde group or keto group or hemiacetal group, to a reaction wherein the carboxy group is transformed into a reactive carboxy group, and the resulting at least bifunctional compound is purified and reacted with functional group Q of the polymer derivative.

Specific examples of the at least bifunctional compound comprising a carboxy group which may be reacted to obtain a reactive carboxy group are the compounds 1 to 11 of the list hereinabove. In this context, the term “carboxy group” also relates to a lacton and an internal anhydride of a dicarboxylic acid compound.

Thus, according to a preferred embodiment, the present invention relates to a method and a conjugate as described above, wherein the polymer derivative comprising Q, Q being an amino group —NH₂, or a derivative of the amino group comprising the structure unit —NH-such as aminoalkyl groups, aminoaryl group, aminoaralkyl groups, or alkarylamino groups, is further reacted with formylbenzoic acid.

According to another embodiment, the present invention relates to a method and a conjugate as described above, wherein the polymer derivative comprising Q, Q being an amino group, is further reacted with formylbenzoic acid pentafluorophenyl ester.

According to yet another embodiment, the present invention relates to a method and a conjugate as described above, wherein the polymer derivative comprising Q, Q being an amino group, is further reacted with formylbenzoic acid N-hydroxysuccinimide ester.

According to yet another embodiment, the present invention relates to a method and a conjugate as described above, wherein the polymer derivative comprising Q, Q being an amino group, is further reacted with 4-(4-formyl-3,5-dimethoxyphenoxy)butyric acid.

According to another preferred embodiment, the present invention relates to a method and a conjugate as described above, wherein the polymer derivative comprising Q, Q being an amino group —NH₂, is reacted with a bifunctional compound which is a biocompatible compound selected from the group consisting of alpha-keto carboxylic acids, sialic acids or derivatives thereof and pyridoxal phosphate.

As regards alpha-keto carboxylic acids, those are preferably alpha-keto carboxylic acids derived from amino acids and can in most instances also be found in the human body. Preferred alpha-keto carboxylic acids derived from amino acids are selected from the group consisting of keto-valine, keto-leucine, keto-isoleucine and keto-alanine. The carboxy group of the alpha-keto carboxylic acids is reacted with group Q of the polymer being an amino group. Therewith an amido group is formed. The remaining free keto group of the alpha-keto carboxylic acid may then be treated with a functional group of the protein, in particular an amino group. Therewith an imino group is formed which may be hydrogenated.

Accordingly, the present invention relates to a method and a conjugate as described above, wherein the polymer derivative comprising Q, Q being an amino group, is reacted with an alpha-keto carboxylic acid.

As regards sialic acids or derivatives thereof those are preferably biocompatible, in particular they are sugars found in the human body, which are N- and/or O-acetylated. In a preferred embodiment, sialic acids are N-acetyl neuramic acids. These compounds show a desired rigidity because of the pyranose structure in order to fulfill the function as a spacer. On the other hand, it may be possible to introduce an aldehyde group into these compounds through selective oxidation. Sialic acids are found in the human body e.g. as terminal monosaccarides in glycan chains of glycosylated proteins.

In a preferred embodiment, the sialic acid may be selectively oxidized to an aldehyde group.

Methods to selectively oxidize sialic acids are known in the art, e.g. from L. W. Jaques, B.F. Riesco, W. Weltner, Carbohydrate Research, 83 (1980), 21-32 and T. Masuda, S. Shibuya, M. Arai, S. Yoshida, T. Tomozawa, A. Ohno, M. Yamashita, T. Honda, Bioorganic & Medicinal Chemistry Letters, 13 (2003), 669-673. Preferably the oxidation of the sialic acid may be conducted prior to the reaction with the amino group of the polymer.

The optionally oxidized sialic acid, may then be reacted via its carboxylic acid group with the amino group of the polymer.

The resulting compounds contain an aldehyde group which can then further be reacted by reductive amination with an amino group of a protein.

Accordingly, the present invention relates to a method and a conjugate as described above, wherein the polymer derivative comprising Q, Q being an amino group, is reacted with an optionally oxidized sialic acid.

As regards pyridoxal phosphate (PyP), this is a highly biocompatible bifunctional compound and is also called vitamin B6. PyP is a co-enzyme which participates in transaminations, decarboxylations, racemization, and numerous modifications of amino acid side chains. All PyP requiring enzymes act via the formation of a Schiff's base between the amino acid and the co-enzyme.

The phosphate group of the PyP may be reacted with the amino group of the polymer, preferably hydroxyalkyl starch, in particular hydroxyethyl starch, forming a phosphoramide. The aldehyde group of PyP may then be reacted with the amino group of a protein, forming a Schiff's base, which may then be reduced. In a preferred embodiment, the structure of the conjugate is HES-NH—P(O)₂—O-(pyridoxal)-CH—NH-protein.

In case of PyP, the functional group of the polymer is preferably introduced into the polymer by use of a di-amino compound as described above.

Accordingly, the present invention relates to a method and a conjugate as described above, wherein the polymer derivative comprising Q, Q being an amino group, is reacted with pyridoxal phosphate.

As solvent for the reaction of the polymer derivative comprising an amino group and, e.g., formylbenzoic acid, at least one aprotic solvent or at least one polar solvent is preferred. Suitable solvents are, among others, water, dimethyl sulfoxide (DMSO), N-methyl pyrrolidone, dimethyl acetamide (DMA), dimethyl formamide (DMF) and mixtures of two or more thereof.

As solvent for the reaction of the polymer derivative comprising an amino group and the at least bifunctional compound comprising a carboxy group, it is also possible to use an aqueous medium. The term “aqueous medium” as used in this context of the present invention relates to a solvent or a mixture of solvents comprising water in the range of from at least 10% per weight or at least 20% per weight or at least 30% per weight or at least 40% per weight or at least 50% per weight or at least 60% per weight or at least 70% per weight or at least 80% per weight or at least 90% per weight or up to 100% per weight, based on the weight of the solvents involved.

The reaction is preferably carried out at a temperature of from 0 to 40° C., more preferably of from 0 to 25° C. and especially preferably of from 15 to 25° C. for a reaction time preferably of from 0.5 to 24 h and especially preferably of from 1 to 17 h.

According to a preferred embodiment, the reaction is carried out in the presence of an activating agent. Suitable activating agents are, among others, carbodiimides such as diisopropyl carbodiimde (DIC), dicyclohexyl carbodiimides (DCC), 1-ethyl-3-(3-dimethylaminopropyl)carbodiimide (EDC), with diisopropyl carbodiimde (DIC) being especially preferred.

The resulting polymer derivative may be purified from the reaction mixture by at least one suitable method. If necessary, the polymer derivative may be precipitated prior to the isolation by at least one suitable method.

If the polymer derivative is precipitated first, it is possible, e.g., to contact the reaction mixture with at least one solvent or solvent mixture other than the solvent or solvent mixture present in the reaction mixture at suitable temperatures. According to a particularly preferred embodiment of the present invention where an aqueous medium, preferably water is used as solvent, the reaction mixture is contacted with 2-propanol or with am mixture of acetone and ethanol, preferably a 1:1 mixture (v/v), indicating equal volumes of said compounds, at a temperature, preferably in the range of from −20 to +50° C. and especially preferably in the range of from −20 to 25° C.

Isolation of the polymer derivative may be carried out by a suitable process which may comprise one or more steps. According to a preferred embodiment of the present invention, the polymer derivative is first separated off the reaction mixture or the mixture of the reaction mixture with, e.g., aqueous 2-propanol mixture, by a suitable method such as centrifugation or filtration. In a second step, the separated polymer derivative may be subjected to a further treatment such as an after-treatment like dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first dialysed, preferably against water, and then lyophilized until the solvent content of the reaction product is sufficiently low according to the desired specifications of the product. Lyophilisation may be carried out at temperature of from 20 to 35° C., preferably of from 20 to 30° C.

The resulting polymer derivative with the aldehyde group or keto group or hemiacetal group is subsequently reacted with an amino group of the protein via reductive amination.

The reductive amination reaction according to the invention, wherein the polymer or polymer derivative is covalently linked via at least one aldehyde group or keto group or hemiacetal group to at least one amino group of the modified glycoprotein, preferably the amino group introduced as functional group Z during step a) of the method of the invention, is preferably carried out at a temperature of from 0 to 40° C., more preferably 0 to 37° C., more preferably of from 0 to 25° C., in particular from 4 to 21° C., but especially preferably of from 0 to 21° C. The reaction time preferably ranges of from 0.5 to 72 h, more preferably of from 2 to 48 h and especially preferably of from 4 to 7 h. As solvent for the reaction, an aqueous medium is preferred.

Thus, the present invention also relates to a method and a conjugate as described above, wherein the reductive amination is carried out at a temperature of from 0 to 21° C.

Therefore, the present invention also relates to a method and a conjugate as described above, wherein reductive amination is carried out in an aqueous medium.

Thus, the present invention also relates to a method and conjugate as described above, wherein the reductive amination is carried out at a temperature of from 0 to 21° C. in an aqueous medium.

The term “aqueous medium” as used in the context of the present invention relates to a solvent or a mixture of solvents comprising water in the range of from at least 10% per weight, more preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The preferred reaction medium is water.

The pH value of the reaction medium is generally in the range of from 4 to 9 or from 4 to 8 or from 4 to 7.3. According to a preferred embodiment of the present invention, the pH at which the reductive amination reaction is carried out is below 10, preferably below 7.5, preferably 7.3, more preferably smaller or equal 7 and most preferably below 7, i.e. in the acidic range. Preferred ranges are therefore of from 3 to below 7, more preferably of from 3.5 to 6.5, still more preferably of from 4 to 6, still more preferably of from 4.5 to 5.5 and especially preferably about 5.0, i.e. 4.6 or 4.7 or 4.8 or 4.9 or 5.0 or 5.1 or 5.2 or 5.3 or 5.4. Preferred ranges, are among others, 3 to 6.9 or 3 to 6.5 or 3 to 6 or 3 to 5.5 or 3 to 5 or 3 to 4.5 or 3 to 4 or 3 to 3.5 or 3.5 to 6.9 or 3.5 to 6.5 or 3.5 to 6 or 3.5 to 5.5 or 3.5 to 5 or 3.5 to 4.5 or 3.5 to 4 or 4 to 6.9 or 4 to 6.5 or 4 to 6 or 4 to 5.5 or 4 to 5 or 4 to 4.5 or 4.5 to 6.9 or 4.5 to 6.5 or 4.5 to 6 or 4.5 to 5.5 or 4.5 to 5 or 5 to 6.9 or 5 to 6.5 or 5 to 6 or 5 to 5.5 or 5.5 to 6.9 or 5.5 to 6.5 or 5.5 to 6 or 6 to 6.9 or 6 to 6.5 or 6.5 to 6.9.

The molar ratio of polymer derivative: protein used for the reaction is preferably in the range of from 200:1 to 5:1, more preferably of from 100:1 to 10:1 and especially preferably of from 75:1 to 20:1.

The invention also relates to the embodiments as described hereinabove, wherein the position of groups Z and A is reversed, in particular, wherein the functional group introduced into the glycoprotein during step a) of the method of the invention is a group containing a reactive carboxy group or an aldehyde group, a hemiacetal group or a keto group and wherein the reactive group A of the polymer or polymer derivative is amino group. It is particularly preferred that the group A is selected from a hydroxylamine, a hydrazine, a hydrazid or a hydrazide derivative as described above.

According to another preferred embodiment of the present invention, the functional group Z of the modified polyol to be reacted with functional group A of the polymer or polymer derivative is a thiol group. Preferably, the glycoprotein is selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX.

The thiol group is introduced in step a) by use of a thiol-modified polyol in the transferase reaction.

According to a first embodiment, the functional group Z of the modified polyol is a thiol group and functional group A of the polymer is a halogenacetyl group and wherein A is introduced by reacting the polymer at its optionally oxidized reducing end with an at least bifunctional compound having at least two functional groups each comprising an amino group to give a polymer derivative having at least one functional group comprising an amino group and reacting the polymer derivative with a monohalogen-substituted acetic acid and/or a reactive monohalogen-substituted acetic acid derivative.

As to the at least bifunctional compound having at least two functional groups each comprising an amino group, all compounds are conceivable which are capable of being reacted with the polymer at its optionally reducing end to give a polymer derivative comprising an amino group which can be reacted with a monohalogen-substituted acetic acid and/or a reactive monohalogen-substituted acetic acid derivative.

According to a preferred embodiment, one functional group of the at least bifunctional compound, said functional group being reacted with the optionally oxidized reducing end of the polymer, is selected from the group consisting of

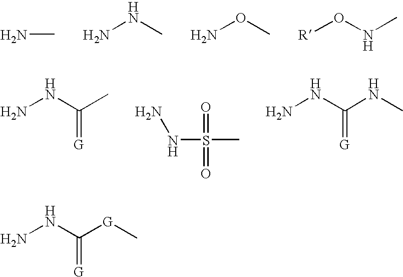

wherein G is O or S and, if present twice, independently O or S, and R′ is methyl.

According to an especially preferred embodiment of the present invention, the functional group of the at least bifunctional compound, said functional group being reacted with the optionally oxidized reducing end, is the amino group —NH₂. According to a still further preferred embodiment, this functional group, most preferably the amino group, is reacted with the oxidized reducing end of the polymer.

According to a preferred embodiment of the present invention, the functional group of the at least bifunctional compound, said functional group being reacted with the monohalogen-substituted acetic acid and/or a reactive monohalogen-substituted acetic acid derivative, is an amino group —NH₂.

The functional groups, preferably both being an amino group —NH₂, of the at least bifunctional compound, said functional groups being reacted with the polymer at its optionally oxidized reducing end, preferably the oxidized reducing end, and the monohalogen-substituted acetic acid and/or a reactive monohalogen-substituted acetic acid derivative, may be separated by any suitable spacer. Among others, the spacer may be an optionally substituted, linear, branched and/or cyclic hydrocarbon residue. Suitable substituents are, among others, alkyl, aryl, aralkyl, alkaryl, halogen, carbonyl, acyl, carboxy, carboxyester, hydroxy, thio, alkoxy and/or alkylthio groups. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 2 to 10, and especially preferably from 2 to 8 carbon atoms. Thus, preferred at least bifunctional compounds are bifunctional amino compounds, especially preferably 1,8-diamino octane, 1,7-diamino heptane, 1,6-diamino hexane, 1,5-diamino pentane, 1,4-diamino butane, 1,3-diamino propane, and 1,2-diamino ethane. According to a further preferred embodiment, the at least bifunctional compound is a diaminopolyethylenglycol, preferably a diaminopolyethylenglycol according to formula

H₂N—(CH₂—CH₂—O)ₘ—CH₂—CH₂—NH₂

wherein m is an integer, m preferably being 1, 2, 3, or 4.

Therefore, the present invention also relates to a method and a conjugate as described above, wherein the polymer is reacted with 1,8-diaminooctane, 1,7-diaminoheptane, 1,6-diaminohexane, 1,5-diaminopentane, 1,4-diaminobutane, 1,3-diaminopropane, and 1,2-diaminoethane at its oxidized reducing end with to give a polymer derivative according to the formula

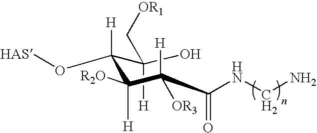

with n=2, 3, 4, 5, 6, 7, or 8, and the polymer especially preferably being HES.

Therefore, the present invention also relates to a method and a conjugate as described above, wherein the polymer is reacted with H₂N—(CH₂—CH₂—O)ₘ—CH₂—CH₂—NH₂ at its oxidized reducing end, wherein m is 1, 2, 3, or 4, to give a polymer derivative according to the formula

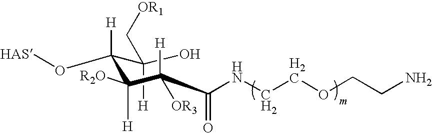

with m=1, 2, 3, or 4, and the polymer especially preferably being HES.

The oxidation of the reducing end of the polymer, preferably hydroxyethyl starch, may be carried out according to each method or combination of methods which result in compounds having the structures (IIa) and/or (IIb):

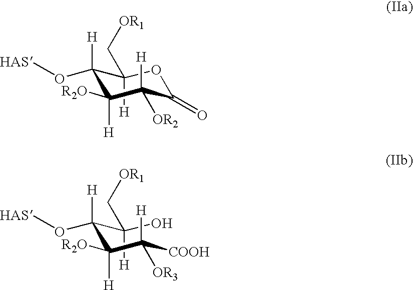

Although the oxidation may be carried out according to all suitable method or methods resulting in the oxidized reducing end of hydroxyalkyl starch, it is preferably carried out using an alkaline iodine solution as described, e.g., in DE 196 28 705 A1 the respective contents of which (example A, column 9, lines 6 to 24) is incorporated herein by reference.

The polymer derivative resulting from the reaction of the polymer with the at least bifunctional compound, is further reacted with the monohalogen-substituted acetic acid and/or a reactive monohalogen-substituted acetic acid derivative.

As monohalogen-substituted acetic acid or reactive acid, Cl-substituted, Br-substituted and I-substituted acetic acid are preferred.

If the halogen-substituted acid is employed as such, it is preferred to react the acid with the polymer derivative in the presence of an activating agent. Suitable activating agents are, among others, Suitable activating agents are, among others, carbodiimides such as diisopropyl carbodiimde (DIC), dicyclohexyl carbodiimides (DCC), 1-ethyl-3-(3-dimethylaminopropyl)carbodiimide (EDC), with dicyclohexyl carbodiimides (DCC) and 1-ethyl-3-(3-dimethylaminopropyl)carbodiimide (EDC) being especially preferred.

Therefore, the present invention also relates to a method and a conjugate as described above, wherein the polymer, preferably HES, is reacted with a diamino compound, preferably a diaminoalkane with 2 to 8 carbon atoms or H₂N—(CH₂—CH₂—O)ₘ—CH₂—CH₂—NH₂ with m=1, 2, 3, or 4, and reacting the resulting polymer derivative with Br-substituted and I-substituted acetic acid in the presence of an activating agent, preferably EDC.

Therefore, the present invention also relates to a polymer derivative according to the formula

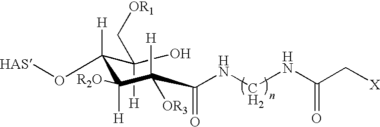

with X═Cl, Br or I, n=2, 3, 4, 5, 6, 7, or 8, and the polymer especially preferably being HES, or a polymer derivative according to the formula

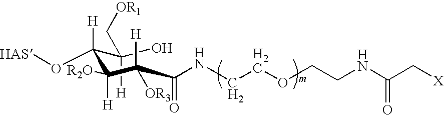

with X═Cl, Br or I, m=1, 2, 3, or 4, and the polymer especially preferably being HES.

The reaction of the polymer derivative with the halogen-substituted acetic acid is preferably carried out it in DMF or an aqueous system, preferably water, at a preferred pH of from 3.5 to 5.5, more preferably of 4.0 to 5.0 and especially preferably from 4.5 to 5.0; and a preferred reaction temperature of from 4 to 30° C., more preferably from 15 to 25° C. and especially preferably from 20 to 25° C.; and for a preferred reaction time of from 1 to 8 h, more preferably from 2 to 6 h and especially preferably from 3 to 5 h.

The reaction mixture comprising the polymer derivative which comprises the polymer, the at least bifunctional compound and the halogen-substituted acetic acid, can be used for the reaction with the modified glycoprotein obtained from step a) as such. According to a preferred embodiment of the present invention, the polymer derivative is separated from the reaction mixture, preferably by ultrafiltration, subsequent precipitation, optional washing and drying in vacuo.

The reaction of the polymer derivative with the protein is carried out at a preferred pH of from 6.5 to 8.5, more preferably from 7.0 to 8.5 and especially preferably from 7.5 to 8.5; and a preferred reaction temperature of from 0 to 40° C., more preferably from 1 to 25° C. and especially preferably from 15 to 25° C. or alternatively from 1 to 15° C.; and for a preferred reaction time of from 0.5 to 8 h, more preferably from 1 to 6 h and especially preferably from 2 to 5 h.

The reaction of the polymer derivative with the thiol group of the modified polyol results in a thioether linkage between the polymer derivative and the modified polyol attached to the glycoprotein.

Therefore, the present invention also relates to a method and a conjugate as described above, wherein the polymer, preferably HES, is reacted with a diamino compound, preferably a diaminoalkane with 2 to 8 carbon atoms or H₂N—(CH₂—CH₂—O)ₙ—CH₂—CH₂—NH₂ with m=1, 2, 3, or 4, the resulting polymer derivative is reacted with Br-substituted and I-substituted acetic acid in the presence of an activating agent, preferably EDC, and the resulting polymer derivative is reacted with a thiol group of the protein to give a conjugate comprising a thioether linkage between the protein and the polymer derivative.

Therefore, the present invention also relates to a conjugate according to the formula

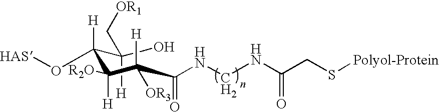

with n=2, 3, 4, 5, 6, 7, or 8, and the polymer especially preferably being HES and the glycoprotein being selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX, the S atom being derived from the modified polyol.

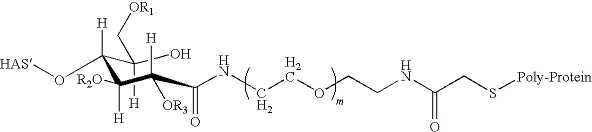

with m=1, 2, 3, or 4, and the polymer especially preferably being HES and the glycoprotein being selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX, the S atom being derived from the modified polyol.

The hydroxyethyl starch is preferably hydroxyethyl starch having a mean molecular weight of about 10 kD and a DS of about 0.4 or hydroxyethyl starch having a mean molecular weight of about 10 kD and a DS of about 0.7 or hydroxyethyl starch having a mean molecular weight of about 12 kD and a DS of about 0.4 or hydroxyethyl starch having a mean molecular weight of about 12 kD and a DS of about 0.7 or hydroxyethyl starch having a mean molecular weight of about 18 kD and a DS of about 0.4 or hydroxyethyl starch having a mean molecular weight of about 18 kD and a DS of about 0.7 or hydroxyethyl starch having a mean molecular weight of about 30 kD and a DS of about 0.4 or hydroxyethyl starch having a mean molecular weight of about 30 kD and a DS of about 0.7, or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

According to a second embodiment, functional group Z of the modified polyol is a thiol group and functional group A of the polymer comprises a maleimido group.

According to this embodiment, several possibilities exist to produce the conjugate. In general, the polymer is reacted at its optionally oxidized reducing end with at least one at least bifunctional compound, wherein this at least bifunctional compound comprises one functional group which is capable of being reacted with the optionally oxidized reducing end of the polymer, and at least one functional group which either comprises the maleimido group or is chemically modified to give a polymer derivative which comprises the maleimido group. According to a preferred embodiment, said functional group is chemically modified to give a polymer derivative which comprises the maleimido group.

Therefore, the present invention relates to a method and a conjugate as described above, by reacting a polymer derivative comprising a maleimido group with a thiol group of the modified polyol, said method comprising reacting the polymer at its optionally oxidized reducing end with an at least bifunctional compound comprising a functional group U capable of reacting with the optionally oxidised reducing end, the at least bifunctional compound further comprising a functional group W capable of being chemically modified to give a maleimido group, the method further comprising chemically modifying the functional group W to give a maleimido group.

As to functional group U, each functional group is conceivable which is capable of being reacted with optionally oxidised reducing end of the polymer.

According to a preferred embodiment of the present invention, the functional group U comprises the chemical structure —NH—.

Therefore, the present invention also relates to a method and a conjugate as described above, wherein the functional group U comprises the structure —NH—.

According to one preferred embodiment of the present invention, the functional group U is a group having the structure R′—NH— where R′ is hydrogen or a alkyl, cycloalkyl, aryl, aralkyl, arylcycloalkyl, alkaryl or cycloalkylaryl residue where the cycloalkyl, aryl, aralkyl, arylcycloalkyl, alkaryl or cycloalkylaryl residue may be linked directly to the NH group or, according to another embodiment, may be linked by an oxygen bridge to the NH group. The alkyl, cycloalkyl, aryl, aralkyl, arylcycloalkyl, alkaryl, or cycloalkylaryl residues may be suitably substituted. As preferred substituents, halogenes such as F, Cl or Br may be mentioned. Especially preferred residues R′ are hydrogen, alkyl and alkoxy groups, and even more preferred are hydrogen and unsubstituted alkyl and alkoxy groups.

Among the alkyl and alkoxy groups, groups with 1, 2, 3, 4, 5, or 6 C atoms are preferred. More preferred are methyl, ethyl, propyl, isopropyl, methoxy, ethoxy, propoxy, and isopropoxy groups. Especially preferred are methyl, ethyl, methoxy, ethoxy, and particular preference is given to methyl or methoxy.

Therefore, the present invention also relates to a method and a conjugate as described above, wherein R′ is hydrogen or a methyl or a methoxy group.

According to another preferred embodiment of the present invention, the functional group U has the structure R′—NH—R″—where R″ preferably comprises the structure unit —NH— and/or the structure unit —(C=G)- where G is O or S, and/or the structure unit —SO₂—. According to more preferred embodiments, the functional group R″ is selected from the group consisting of

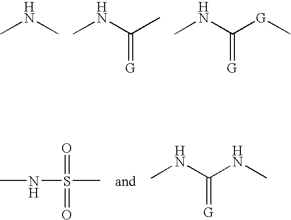

where, if G is present twice, it is independently O or S.

Therefore, the present invention also relates to a method and a conjugate as described above, wherein the functional group U is selected from the group consisting of

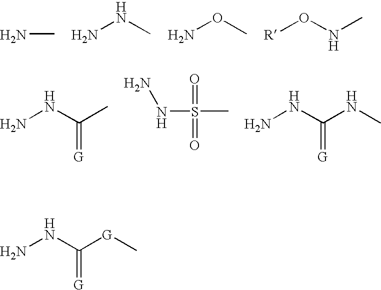

wherein G is O or S and, if present twice, independently O or S, and R′ is methyl.

According to a still more preferred embodiment of the present invention, U comprises an amino group —NH₂.

According to an embodiment of the present invention, the functional group W of the at least bifunctional compound is chemically modified by reacting the polymer derivative comprising W with a further at least bifunctional compound comprising a functional group capable of being reacted with W and further comprising a maleimido group.

As to functional group W and the functional group of said further at least bifunctional compound which is capable of being reacted with W, the following functional groups are to be mentioned, among others:
C—C-double bonds or C—C-triple bonds or aromatic C—C-bonds;
the thio group or the hydroxy groups;
alkyl sulfonic acid hydrazide, aryl sulfonic acid hydrazide;
1,2-dioles;
1,2-aminoalcohols;
1,2 amino-thioalcohols;
the amino group —NH₂ or derivatives of the amino groups comprising the structure unit —NH— such as aminoalkyl groups, aminoaryl group, aminoaralkyl groups, or alkarylamino groups;
the hydroxylamino group —O—NH₂, or derivatives of the hydroxylamino group comprising the structure unit —O—NH—, such as hydroxylalkylamino groups, hydroxylarylamino groups, hydroxylaralkylamino groups, or hydroxylalkarylamino groups;
alkoxyamino groups, aryloxyamino groups, aralkyloxyamino groups, or alkaryloxyamino groups, each comprising the structure unit —NH—O—;
residues having a carbonyl group, -Q-C(=G)-M, wherein G is O or S, and M is, for example,
   —OH or —SH;
   an alkoxy group, an aryloxy group, an aralkyloxy group, or an alkaryloxy group;
   an alkylthio group, an arylthio group, an aralkylthio group, or an alkarylthio group;
   an alkylcarbonyloxy group, an arylcarbonyloxy group, an aralkylcarbonyloxy group, an alkarylcarbonyloxy group;
   activated esters such as esters of hydroxylamines having imid structure such as N-hydroxysuccinimide or having a structure unit O—N where N is part of a heteroaryl compound or, with G=O and Q absent, such as aryloxy compounds with a substituted aryl residue such as pentafluorophenyl, paranitrophenyl or trichlorophenyl;
wherein Q is absent or NH or a heteroatom such as S or O;
—NH—NH₂, or —NH—NH—;
—NO₂;
the nitril group;
carbonyl groups such as the aldehyde group or the keto group;
the carboxy group;
the —N═C═O group or the —N═C═S group;
vinyl halide groups such as the vinyl iodide or the vinyl bromide group or triflate;
—C≡C—H;
—(C═NH₂Cl)-OAlkyl
groups —(C═O)—CH₂-Hal wherein Hal is Cl, Br, or I;
—CH═CH—SO₂—;
a disulfide group comprising the structure —S—S—;
the group

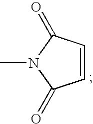

the group

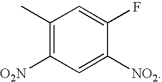

where W and the functional group of the further at least bifunctional compound, respectively, is a group capable of forming a chemical linkage with one of the above-mentioned groups.

According to a still more preferred embodiment of the present invention, W comprises an amino group —NH₂.

According to preferred embodiments of the present invention, both W and the other functional group are groups from the list of groups given above.

According to one embodiment of the present invention, one of these functional groups is a thio group. In this particular case, the other functional group is preferably selected from the group consisting of

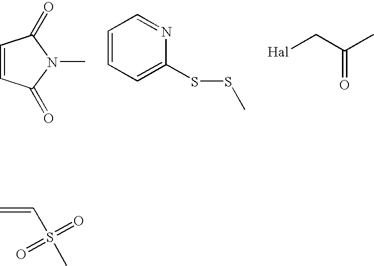

wherein Hal is Cl, Br, or I, preferably Br or I.

According to an especially preferred embodiment of the present invention, one of these functional groups is selected from the group consisting of a reactive ester such as an ester of hydroxylamines having imide structure such as N-hydroxysuccinimide or having a structure unit O—N where N is part of a heteroaryl compound or such as an aryloxy compound with a substituted aryl residue such as pentafluorophenyl, paranitrophenyl or trichlorophenyl, or a carboxy group which is optionally transformed into a reactive ester. In this particular case, the other functional group comprises the chemical structure —NH—.

According to an especially preferred embodiment of the present invention, W comprises the structure —NH— and the further at least bifunctional compound comprises a reactive ester and the maleimido group.

As to the functional group W comprising the structure —NH—, reference can be made to the functional group as described above, wherein W may be the same or different from U. According to a preferred embodiment of the present invention, U and W are the same. More preferably, both U and W comprise an amino group. Particularly preferred, both U and W are an amino group —NH₂.

According to one embodiment of the present invention, the polymer may be reacted with the at least bifunctional compound comprising U and W at its non-oxidized reducing end in an aqueous medium. According to a preferred embodiment where U and W both are an amino group, the reaction is carried out using the polymer with the reducing end in the oxidized form, in at least one aprotic solvent, particularly preferably in an anhydrous aprotic solvent having a water content of not more than 0.5 percent by weight, preferably of not more than 0.1 percent by weight. Suitable solvents are, among others, dimethyl sulfoxide (DMSO), N-methylpyrrolidone, dimethyl acetamide (DMA), dimethyl formamide (DMF) and mixtures of two or more thereof.

Especially in case both U and W are an amino group —NH₂, U and W may be separated by any suitable spacer. Among others, the spacer may be an optionally substituted, linear, branched and/or cyclic hydrocarbon residue. Suitable substituents are, among others, alkyl, aryl, aralkyl, alkaryl, halogen, carbonyl, acyl, carboxy, carboxyester, hydroxy, thio, alkoxy and/or alkylthio groups. Generally, the hydrocarbon residue has from 1 to 60, preferably from 1 to 40, more preferably from 1 to 20, more preferably from 2 to 10, more preferably from 2 to 6 and especially preferably from 2 to 4 carbon atoms. If heteroatoms are present, the separating group comprises generally from 1 to 20, preferably from 1 to 8 and especially preferably from 1 to 4 heteroatoms. The hydrocarbon residue may comprise an optionally branched alkyl chain or an aryl group or a cycloalkyl group having, e.g., from 5 to 7 carbon atoms, or be an aralkyl group, an alkaryl group where the alkyl part may be a linear and/or cyclic alkyl group. According to an even more preferred embodiment, the hydrocarbon residue is an alkyl chain of from 1 to 20, preferably from 2 to 10, more preferably from 2 to 6, and especially preferably from 2 to 4 carbon atoms.

Therefore, the present invention also relates to a method and a conjugate as described above, wherein the polymer is reacted with its oxidized reducing end with 1,4-diaminobutane, 1,3-diaminopropane or 1,2-diaminoethane to give a polymer derivative according to the formula

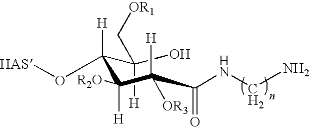

with n=2, 3, or 4, the polymer preferably being HES.

According to the above-mentioned preferred embodiment, the polymer derivative comprising an amino group is further reacted with an at least bifunctional compound comprising a reactive ester group and the maleimido group. The reactive ester group and the maleimido group may be separated by a suitable spacer. As to this spacer, reference can be made to the spacer between the functional groups U and W. According to a preferred embodiment of the present invention, the reactive ester group and the maleimido group are separated by a hydrocarbon chain having from 1 to 10, preferably from 1 to 8, more preferably from 1 to 6, more preferably from 1 to 4, more preferably from 1 to 2 and particularly preferably 1 carbon atom. According to a still further preferred embodiment, the reactive ester is a succinimide ester, and according to a particularly preferred embodiment, the at least bifunctional compound comprising the maleimido group and the reactive ester group is N-(alpha-maleimidoacetoxy)succinimide ester.

Therefore, the present invent also relates to a polymer derivative according to the formula

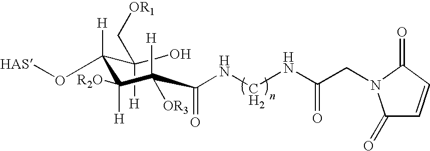

with n=2, 3, or 4, the polymer preferably being HES.

The polymer derivative comprising the maleimido group is further reacted with the thiol group of the modified polyol to give a conjugate comprising the polymer derivative linked to the modified polyol of the modified glycoprotein via a thioether group.

Therefore, the present invention also relates to a conjugate, comprising the glycoprotein, the modified polyol and the polymer, according to the formula

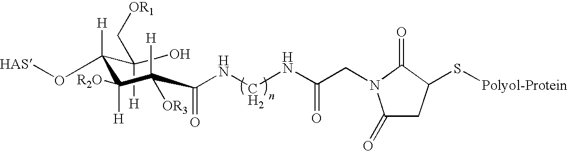

with n=2, 3, or 4, preferably 4, the polymer preferably being HES, the glycoprotein being selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, CSF, factor VII, factor VIII, and factor IX.

The hydroxyethyl starch is preferably hydroxyethyl starch having a mean molecular weight of about 10 kD and a DS of about 0.4 or hydroxyethyl starch having a mean molecular weight of about 10 kD and a DS of about 0.7 or hydroxyethyl starch having a mean molecular weight of about 12 kD and a DS of about 0.4 or hydroxyethyl starch having a mean molecular weight of about 12 kD and a DS of about 0.7 or hydroxyethyl starch having a mean molecular weight of about 18 kD and a DS of about 0.4 or hydroxyethyl starch having a mean molecular weight of about 18 kD and a DS of about 0.7 or hydroxyethyl starch having a mean molecular weight of about 30 kD and a DS of about 0.4 or hydroxyethyl starch having a mean molecular weight of about 30 kD and a DS of about 0.7 or hydroxyethyl starch having a mean molecular weight of about 50 kD and a DS of about 0.4 or hydroxyethyl starch having a mean molecular weight of about 50 kD and a DS of about 0.7 or hydroxyethyl starch having a mean molecular weight of about 100 kD and a DS of about 0.7. As to each of these combinations of mean molecular weight and DS, also a DS value of about 0.8 is preferred.

The reaction of the polymer derivative comprising the maleimido group with the thiol group of the protein is preferably carried in a buffered aqueous system, at a preferred pH of from 5.5 to 8.5, more preferably from 6 to 8 and especially preferably from 6.5 to 7.5, and a preferred reaction temperature of from 0 to 40° C., more preferably from 1 to 25° C. and especially preferably from 15 to 25° C. or alternatively from 1 to 15° C., and for a preferred reaction time of from 0.5 to 24 h, more preferably from 1 to 20 h and especially from 2 to 17 h. The suitable pH value of the reaction mixture may be adjusted by adding at least one suitable buffer. Among the preferred buffers, sodium acetate buffer, phosphate or borate buffers may be mentioned.

The conjugate may be subjected to a further treatment such as an after-treatment like dialysis, centrifugal filtration or pressure filtration, ion exchange chromatography, reversed phase chromatography, HPLC, MPLC, gel filtration and/or lyophilisation.

The present invention also relates to a conjugate, comprising a glycoprotein and a polymer or a derivative thereof, wherein the polymer is a hydroxyalkyl starch (HAS) and the glycoprotein is glycoprotein being selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, CSF, factor VII, factor VIII, and factor IX, said conjugate having a structure according to the formula

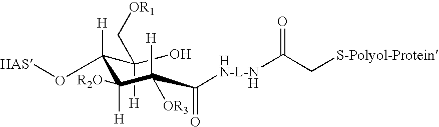

wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, preferably hydrogen or a hydroxyalkyl group, more preferably hydrogen or a hydroxyethyl group, and
wherein L is an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 2 to 60 preferably from 2 to 40, more preferably from 2 to 20, more preferably from 2 to 10 carbon atoms, and
wherein the sulfur atom is derived from a thiol group of a modified polyol.

The present invention also relates to a conjugate as described above, wherein -L- is

—[(CRₐR_{b})ₘG]ₙ[CR_{c}R_{d}]ₒ—
wherein Rₐ; R_{b}, R_{c}, R_{d} are independently hydrogen, alkyl, aryl, preferably hydrogen,
wherein G is selected from the group consisting of O and S, preferably O, and wherein
m 1, 2, 3 or 4, most preferably 2, wherein the residues Rₐ and R_{b} may be the same or different in the m groups (CRₐR_{b});
n 1 to 20, preferably 1 to 10, most preferably 1, 2, 3, or 4;
o 1 to 20, preferably 1 to 10, more preferably 1, 2, 3, 4, 5, more preferably 1 or 2, most preferably 1, wherein the residues R_{c} and R_{d} may be the same or different in the o groups CR_{c}R_{d};
or
wherein
n 0, and
o 2 to 20, preferably 2 to 10, more preferably 2, 3, 4, 5, 6, 7, or 8, wherein the residues R_{c} and R_{d} may be the same or different in the o groups CR_{c}R_{d}.

The present invention also relates to a conjugate, comprising a glycoprotein, a modified polyol and a polymer or a derivative thereof, wherein the polymer is a hydroxyalkyl starch (HAS) and the glycoprotein is glycoprotein being selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX, said conjugate having a structure according to the formula

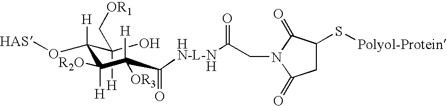

wherein R₁, R₂ and R₃ are independently hydrogen or a hydroxyalkyl group, a hydroxyaryl group, a hydroxyaralkyl group or a hydroxyalkaryl group having of from 2 to 10 carbon atoms, preferably hydrogen or a hydroxyalkyl group, more preferably hydrogen or a hydroxyethyl group, and
wherein L is an optionally substituted, linear, branched and/or cyclic hydrocarbon residue, optionally comprising at least one heteroatom, comprising an alkyl, aryl, aralkyl heteroalkyl, and/or heteroaralkyl moiety, said residue having from 2 to 60 preferably from 2 to 40, more preferably from 2 to 20, more preferably from 2 to 10 carbon atoms, and
wherein the sulfur atom is derived from a thiol group of a modified polyol.

The present invention also relates to a conjugate as described above, wherein -L- is

—[(CRₐR_{b})ₘG]ₙ[(CR_{c}R_{d}]ₒ—
wherein Rₐ; R_{b}, R_{c}, R_{d} are independently hydrogen, alkyl, aryl, preferably hydrogen, wherein G is selected from the group consisting of O and S, preferably O, and wherein
m 1, 2, 3 or 4, most preferably 2, wherein the residues Rₐ and R_{b} may be the same or different in the m groups (CRₐR_{b});
n 1 to 20, preferably 1 to 10, most preferably 1, 2, 3, or 4;
o 1 to 20, preferably 1 to 10, more preferably 1, 2, 3, 4, 5, more preferably 1 or 2, most preferably 1, wherein the residues R_{c} and R_{d} may be the same or different in the o groups CR_{c}R_{d};
Or
wherein
n 0, and
o 2 to 20, preferably 2 to 10, more preferably 2, 3, 4, 5, 6, 7, or 8, wherein the residues R_{c} and R_{d} may be the same or different in the o groups CR_{c}R_{d}.

The present invention also relates to a conjugate as described above, wherein the hydroxyalkyl starch is hydroxyethyl starch.

The present invention also relates to a conjugate as described above, wherein the hydroxyethyl starch has a molecular weight of from 2 to 200 kD, preferably of from 4 to 130 kD, more preferably of from 4 to 70 kD.

The invention also relates to the embodiments as described hereinabove, wherein the position of groups Z and A is reversed, that is, wherein the functional group Z introduced into the glycoprotein during step a) of the method of the invention is a group containing an maleimido group or a halogenacetyl group and wherein the reactive group A of the polymer or polymer derivative is a thiol group.

According to another embodiment of the invention, the functional group Z of the modified polyol is a maleimido group and functional group A of the polymer comprises a thiol group.

According to this embodiment, several possibilities exist to produce the conjugate. In general, the polymer is reacted at its optionally oxidized reducing end with at least one bifunctional compound, wherein this at least bifunctional compound comprises one functional group which is capable of being reacted with the optionally oxidized reducing end of the polymer, and at least one functional group A which comprises the thiol group. Examples for bifunctional compounds useful for this embodiment may be selected from the group consisting of

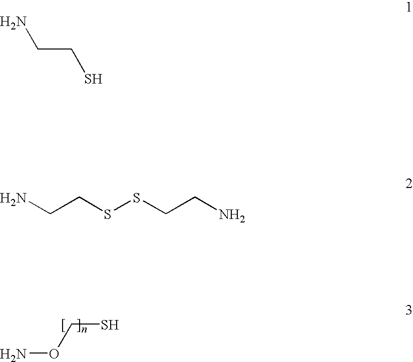

wherein n is an integer, preferably 1, 2, 3, 4, 5, or 6. If the polymer, preferably HES is reacted with compound 3, the covalent linkage formed will be an oxime as already described above. If the polymer, preferably HES, is reacted with compound 1, it is preferably reacted via a reductive amination, as described above. Alternatively, optionally selectively, oxidized polymer, preferably HES, can be reacted with compound 1 whereby a lactone ring opening is conducted. If the polymer, preferably HES is reacted with compound 2 it is preferably reacted via a reductive amination, followed by a cleavage of the S—S— bridge, e.g. with TCEP or DTT. If, optionally selectively, oxidized polymer, preferably HES, is reacted with compound 2, it is preferably reacted via a lactone ring opening, followed by a cleavage of the S—S— bridge, e.g. with TCEP or DTT (For n=2 and n=3 the structure above can be synthesized according to Bauer et al. J. Org. Chem. 1965, 30, 949).

The functional group Z of the of the modified polyol being the maleimido group may be introduced into the modified polyol by any convenient method.

Therefore, the present invention relates to a method and a conjugate as described above, by reacting a polymer derivative comprising a thiol group with a maleimido group of the modified polyol at the protein, said method comprising reacting the polymer at its optionally oxidized reducing end with an at least bifunctional compound comprising a functional group U capable of reacting with the optionally oxidised reducing end, the at least bifunctional compound further comprising a thiol group and reacting the obtained HAS derivative with a protein comprising a maleimido group.

In the methods for preparing a conjugate of the invention the conversion rate in the above described methods may be at least 50%, more preferred at least 70%, even more preferred at least 80% and in particular 95% or even more, such as at least 98% or 99%.

According to a further aspect, the present invention relates to a conjugate as described above, or a conjugate, obtainable by a method as described above, in particular for use in a method for the treatment of the human or animal body.

The conjugates according to the invention may be at least 50% pure, even more preferred at least 70% pure, even more preferred at least 90%, in particular at least 95% or at least 99% pure. In a most preferred embodiment, the conjugates may be 100% pure, i.e. there are no other by-products present.

Therefore, according to another aspect, the present invention also relates to a composition which may comprise the conjugate(s) of the invention, wherein the amount of the conjugate(s) may be at least 50 wt-%, even more preferred at least 70 wt-%, even more preferred at least 90 wt-%, in particular at least 95 wt.-% or at least 99 wt.-%. In a most preferred embodiment, the composition may consist of the conjugate(s), i.e. the amount of the conjugate(s) is 100 wt.-%.

The particularly gentle method of the invention allows to obtain conjugates with very little damage to the glycoprotein part of the conjugate due to oxidation and/or desamidation.

In particular, at least 80% of the glycoproteins comprising at least one free asparagines and/or glutamine side chain retain intact amido groups in the final conjugate with regard to all of the asparagines and glutamine side chains. It is preferred that more than 90%, more than 95% or even more than 99% of the glycoproteins retain all intact amido groups in the final conjugate. It is most preferred that no desamidated glutamine and/or asparagines residues are detectable by mass spectroscopy in the final conjugates. The percentage of desamidated asparagines and/or glutamine residues can be determined by LC-MS according to “Usefulness of Glycopeptide Mapping by Liquid Chromatography/Mass Spectrometry in Comparability Assessment of Glycoprotein Products”, Miyako Ohta, Nana Kawasaki, Satsuki Itoh and Takao Hayakawa, Biologicals Volume 30, Issue 3, September 2002, Pages 235-244.

In particular, at least 80% of the glycoproteins comprising at least one methionine side chain retain their all methionine residues in the non-oxidized form in the final conjugate. It is preferred that more than 90%, more than 95% or even more than 99% of the glycoproteins retain all their methionine residues in the non-oxidized form in the final conjugate. It is most preferred that no oxidized methionine residues are detectable by mass spectroscopy in the final conjugates. The percentage of oxidized methionine residues can be determined by LC-MS according to “Usefulness of Glycopeptide Mapping by Liquid Chromatography/Mass Spectrometry in Comparability Assessment of Glycoprotein Products”, Miyako Ohta, Nana Kawasaki, Satsuki Itoh and Takao Hayakawa, Biologicals Volume 30, Issue 3, September 2002, Pages 235-244.

It is most preferred that both methionine oxidation and glutamine/asparagines desamidation is avoided.

Furthermore, the present invention relates to a pharmaceutical composition comprising in a therapeutically effective amount a conjugate as described above or a conjugate, obtainable by a method as described above.

All glycoprotein-HAS conjugates of the present invention are administered by suitable methods such as e.g. enteral, parenteral or pulmonary methods preferably administered by i.v., s.c. or i.m. routes. The specific route chosen will depend upon the condition being treated. Preferably, the conjugates are administered together with a suitable carrier, such as known in the art (e.g. as used in the first generation/unmodified biopharmaceutical, albumin-free or with albumin as an excipient), a suitable diluent, such as sterile solutions for i.v., i.m., or s.c. application. The required dosage will depend on the severity of the condition being treated, the patients individual response, the method of administration used, and the like. The skilled person is able to establish a correct dosage based on his general knowledge.

According to another aspect, the present invention also relates to the use a HAS-, preferably a HES-protein conjugate as described above or a HAS-, preferably a HES-protein conjugate, obtainable by a method as described above, wherein the protein is Factor VIII, for the preparation of a medicament for the treatment of haemophilia A.

According to another aspect, the present invention also relates to the use of a HAS-AT III conjugate as described above or a HAS-protein conjugate, obtainable by a method as described, for the preparation of a medicament for the treatment of AT III hereditary deficiency, veno-occlusive disease, burns and heparin resistance in coronary arterial bypass Graft (CABG) surgery, bowel perforation resulting from trauma or gastrointestinal surgery; disseminated intravascular coagulation (DIC) and/or sepsis as well as for the prevention of micro-clot formation associated with ventilation therapy. The pharmaceutical composition comprising the HAS-AT III conjugate of the invention may therefore be used for these purposes.

According to another aspect, the present invention also relates to the use a HAS-, preferably a HES-protein conjugate as described above or a HAS-, preferably a HES-protein conjugate, obtainable by a method as described above, wherein the protein is A1AT, for the preparation of a medicament for the treatment of emphysema, cystic fibrosis, atopic dermatitis, and/or bronchitis. The pharmaceutical composition of the invention comprising the HAS-A1AT-conjugate of the invention may also be used for these purposes.

According to another aspect, the present invention also relates to the use a HAS-, preferably a HES-protein conjugate as described above or a HAS-, preferably a HES-protein conjugate, obtainable by a method as described above, wherein the protein is tPA, for the preparation of a medicament for the treatment of myocardial infarctions (heart attacks), thrombosis, thromboembolism or occlusive diseases, especially occlusive arterial diseases.

According to another aspect, the present invention also relates to the use a HAS-, preferably a HES-protein conjugate as described above or a HAS-, preferably a HES-protein conjugate, obtainable by a method as described above, wherein the protein is APC, for the preparation of a medicament for the treatment of severe sepsis, thrombosis, thromboembolism or occlusive diseases, especially occlusive arterial diseases.

According to another aspect, the present invention also relates to the use a HAS-, preferably a HES-protein conjugate as described above or a HAS-, preferably a HES-protein conjugate, obtainable by a method as described above, wherein the protein is IFN alpha, for the preparation of a medicament for the treatment of leukaemia e.g. hairy cell leukaemia, chronic myelogeneous leukaemia, multiple myeloma, follicular lymphoma, cancer, e.g. carcinoid tumour, malignant melanoma and hepatitis, e.g. chronic hepatitis B and chronic hepatitis C.

According to another aspect, the present invention also relates to the use a HAS-, preferably a HES-protein conjugate as described above or a HAS-, preferably a HES-protein conjugate, obtainable by a method as described above, wherein the protein is IFN beta, for the preparation of a medicament for the treatment of multiple sclerosis, preferably relapsing forms of multiple sclerosis.

The invention further relates to the use of a GM-CSF-HAS conjugate as described above, for the preparation of a medicament for myeloid reconstitution following bone marrow transplant or induction chemotherapy in older adults with acute myelogenous leukaemia, bone marrow transplant engraftment failure or delay, mobilization and following transplantation of autologous peripheral blood progenitor cells.

The present invention also relates to the use of a HAS-Factor VII conjugate for the preparation of a medicament for the treatment of episodes in hemophilia A or B patients with inhibitors to Factor VIII or Factor IX.

The present invention also relates to the use of a HAS-Factor IX conjugate for the preparation of a medicament for the control and prevention of hemorrhagic episodes in patients with hemophilia B (e.g. congenital factor IX deficiency or Christmas disease), including control and prevention of bleeding in surgical settings.

The present invention further relates to the use of (a) transferase(s) in the above-mentioned gentle methods for conjugate production, in particular in such methods which avoid enzyme oxidation and/or side-chain desamidation.

The present invention also relates to a second gentle method of conjugate formation between a glycoprotein and a polymer or a polymer derivative. This second gentle method produces a conjugate between a hydroxyalkylstarch (HAS) and a glycoprotein (GPO) by way of the following steps:
a) providing a GPO, which comprises at least one, more preferably at least two terminal galactose residues,
b) oxidizing the terminal galactose residues by the action of the enzyme galactose oxidase (or a related enzyme) to form terminal galactose residues which comprise a reactive aldehydro group,
c) providing modified HAS which is capable of forming a covalent linkage with the aldehydro group obtained in step b), and
d) reacting the GPO of step b) with the HAS of step c). In this way, a HAS-GPO is produced, that comprises one or more HAS molecules per molecule of glycoprotein, wherein each HAS is conjugated to the GPO via a galactose residue of an N-glycan or an O-glycan of the glycoprotein. The glycan of the so obtained conjugates comprise at least one terminal sugar moiety which is not a sialic acid residue.

The GPO having oxidized galactose residues obtained from step b) of this second gentle method is a suitable substrate for step b) of the first gentle method of the invention. The functional group Z in this case is an aldehyde group, and therefore the functional group A of the polymer or the derivative thereof may comprise an amino group according to the structure —NH—. Thus, the GPO having oxidized galactose residues obtained from step b) of this second gentle method can be used in any method wherein Z is an aldehyde, for example as described on pages 21 to 35 herein. The invention relates therefore also to all HAS-GPO conjugates obtainable by using the GPO having oxidized galactose residues obtained from step b) of this second gentle method in step b) of the first gentle method of the invention.

Therefore, the present invention also relates to a method and a conjugate obtainable by any method wherein Z is an aldehyde, for example as described on pages 21 to 35 herein, in particular wherein the functional group A capable of being reacted with the optionally oxidized reducing end of the polymer, comprises an amino group according to structure —NH—.

It is preferred, that the GPO is selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, IFN-γ, CSF, factor VII, factor VIII, and factor IX, preferably the GPO being EPO, in particular EPO having the amino acid sequence of human EPO.

Preferably, the glycosylated EPO is recombinantly produced. This includes the production in eukaryotic cells, preferably mammalian, insect, yeast or in any other cell type which is convenient for the recombinant production of glycosylated EPO. Furthermore, the EPO may be expressed in transgenic animals (e.g. in body fluids like milk, blood, etc.), in eggs of transgenic birds, especially poultry, preferred chicken, or in transgenic plants.

The recombinant production of a glycosylated polypeptide is known in the art. In general, this includes the transfection of host cells with an appropriate expression vector, the cultivation of the host cells under conditions which enable the production of the glycosylated polypeptide and the purification of the polypeptide from the host cells. For detailed information see e.g. Krystal, Pankratz, Farber, Smart, 1986, Purification of human erythropoietin to homogeneity by a rapid five-step procedure, Blood, 67(1), 71-9; Quelle, Caslake, Burkert, Wojchowski, 1989, High-level expression and purification of a recombinant human erythropoietin produced using a baculovirus vector, Blood, 74(2), 652-7; EP 640 619 B1 and EP 668 351 B1.

In a preferred embodiment, the EPO has the amino acid sequence of human EPO (see EP 148 605 B2).

The EPO may comprise one or more carbohydrate side chains (preferably 1-4, preferably 4) attached to the EPO via N- and/or O-linked glycosylation, i.e. the EPO is glycosylated. Usually, when EPO is produced in eukaryotic cells, the polypeptide is posttranslationally glycosylated. Consequently, the carbohydrate side chains may have been attached to the EPO during biosynthesis in mammalian, especially human, insect or yeast cells. It is preferred that the EPO be produced in a host, which is deficient in capping the glycans with terminal sialic acid residues, thereby generating EPO which is high in terminal galactose residues. This undersialylated EPO can be directly used as the starting material in the second gentle method of the invention.

Since EPO is to be taken as an example of the GPO of the conjugate of the invention, of course also undersialylated forms of other GPOs to be used as the starting material for conjugate formation by this second gentle method of the invention can be produced in such hosts. Particularly preferred are the glycoproteins selected from the group consisting of erythropoietin (EPO), IFN beta, G-CSF, GM-CSF, APC, tPA, A1AT, AT III, HCG, LH, FSH, IL-15, an antibody fusion protein, a therapeutic antibody, an interleukin, especially interleukin 2 or 6, IFN-α, CSF, factor VII, factor VIII, and factor IX.

The gentle second method of the invention (and also the combination of steps a and b of the second method of the invention with step b of the first method of the invention) also allows to produce conjugates with very little damage to the glycoprotein part of the conjugate due to oxidation and/or desamidation.

In particular, at least 80% of the glycoproteins comprising at least one free asparagines and/or glutamine side chain retain intact amido groups in the final conjugate with regard to all of the asparagines and glutamine side chains. It is preferred that more than 90%, more than 95% or even more than 99% of the glycoproteins retain all intact amido groups in the final conjugate. It is most preferred that no desamidated glutamine and/or asparagines residues are detectable by mass spectroscopy in the final conjugates. The percentage of desamidated asparagines and/or glutamine residues can be determined by LC-MS according to “Usefulness of Glycopeptide Mapping by Liquid Chromatography/Mass Spectrometry in Comparability Assessment of Glycoprotein Products”, Miyako Ohta, Nana Kawasaki, Satsuki Itoh and Takao Hayakawa, Biologicals Volume 30, Issue 3, September 2002, Pages 235-244.

In particular, at least 80% of the glycoproteins comprising at least one methionine side chain retain their all methionine residues in the non-oxidized form in the final conjugate. It is preferred that more than 90%, more than 95% or even more than 99% of the glycoproteins retain all their methionine residues in the non-oxidized form in the final conjugate. It is most preferred that no oxidized methionine residues are detectable by mass spectroscopy in the final conjugates. The percentage of oxidized methionine residues can be determined by LC-MS according to “Usefulness of Glycopeptide Mapping by Liquid Chromatography/Mass Spectrometry in Comparability Assessment of Glycoprotein Products”, Miyako Ohta, Nana Kawasaki, Satsuki Itoh and Takao Hayakawa, Biologicals Volume 30, Issue 3, September 2002, Pages 235-244.

In the case of EPO, such an examination by peptide mapping is described in Eur. Phar. 4^{th} edition (01/2002: 1316) pages 1123-1128.

It is most preferred that both methionine oxidation and glutamine/asparagines desamidation is avoided.

The HAS may be directly conjugated to the GPO, like EPO or, alternatively, via a linker molecule. Suitable linker molecules are described above.

According to a preferred embodiment of the HAS-GPO conjugate of the invention, the HAS is conjugated to the GPO via the galactose residue of an attached N- or O-glycan.

Furthermore, the HAS-GPO, for example the HAS-EPO can exhibit a greater in vivo activity than the GPO, for example the EPO, used as a starting material for conjugation (unconjugated GPO). Methods for determining the in vivo biological activity are known in the art (see above).

The HAS-GPO, for example the HAS-EPO, conjugate may exhibit an in vivo activity of 110% to 300%, preferably 110% to 200%, more preferred 110% to 180% or 110% to 150%, most preferred 110% to 140%, if the in vivo activity of the unconjugated GPO, for example EPO, is set as 100%.

Compared to the highly sialylated EPO of Amgen (see EP 428 267 B1), the HAS-EPO exhibits preferably at least 50%, more preferred at least 70%, even more preferred at least 85% or at least 95%, at least 150%, at least 200% or at least 300% of the in vivo activity of the highly sialylated EPO, if the in vivo activity of highly sialylated EPO is set as 100%. Most preferred, it exhibits at least 95% of the in vivo activity of the highly sialylated EPO.

Without wishing to be bound by any theory, the high in vivo biological activity of the HAS-EPO conjugate of the invention probably results from the fact that the HAS-EPO conjugate remains longer in the circulation than the unconjugated EPO, because it is less recognized by the removal systems of the liver and because renal clearance is reduced due to the higher molecular weight. Furthermore, the gentle mode of coupling avoids structural damage to the glycoprotein part. Methods for the determination of the in vivo half-life of EPO in the circulation are known in the art (Sytkowski, Lunn, Davis, Feldman, Siekman, 1998, Human erythropoietin dimers with markedly enhanced in vivo activity, Proc. Natl. Acad. Sci. USA, 95(3), 1184-8).

The invention further relates to a HAS-GPO, for example HAS-EPO, according to the invention for use in a method for treatment of the human or animal body.

Furthermore, the present invention relates to a pharmaceutical composition comprising the HAS-GPO, for example the HAS-EPO of the invention. In a preferred embodiment, the pharmaceutical composition comprises further at least one pharmaceutically acceptable diluent, adjuvant and/or carrier useful in erythropoietin therapy. It is preferred that the concentration of the HAS-GPO is above 1 nM. More preferably, the HAS-GPO constitutes more than 5% of the total protein present, preferably more than 10%, more than 15%, more than 20%, more than 25% and even more than 50%.

It is a particular advantage of the present invention that such GPO preparations may be used, which would otherwise be discarded as being unsuitable for use in a pharmaceutical preparation. In particular, the invention relates to the use of a GPO, in particular EPO, having no more than 70% of the in vivo bioactivity compared to the fully sialylated form of said GPO, for example of EPO, as the starting material in a method for the preparation of modified GPO suitable for use in a pharmaceutical composition.

In the case of EPO, this means that even EPO preparations may be used as starting material which show an in vivo bioactivity of below 100 000 U/mg, or even below 60 000 U/mg, such as below 50 000 U/mg, or even below 40 000 U/mg, such as below 30 000 U/mg or below 20 000 U/mg EPO protein or even showing no detectable in vivo bioactivity, as measured by the normocytohaemic mouse system according to the procedures described in the European Pharmacopeia 4, Monography 01/2002:1316.

The GPO-HAS-conjugate of the invention shows a significant increase in in vivo bioactivity as compared to the undersialylated starting GPO. The increase is typically in the range of 1.5-25 fold, more typically in the range of 2-10 fold, but most of the times 3-8 fold. However, it is pointed out that the method of the invention may even convert an undersialylated showing no detectable in vivo bioactivity into a GPO-HAS-conjugate showing a comparable or better in vivo bioactivity than a fully sialylated GPO.

A “therapeutically effective amount” as used herein refers to that amount which provides therapeutic effect for a given condition and administration regimen. The administration of erythropoietin isoforms is preferably by parenteral routes. The specific route chosen will depend upon the condition being treated.

In the case of EPO, the administration of erythropoietin isoforms is preferably done as part of a formulation containing a suitable carrier, such as human serum albumin, a suitable diluent, such as a buffered saline solution, and/or a suitable adjuvant. The required dosage will be in amounts sufficient to raise the hematocrite of patients and will vary depending upon the severity of the condition being treated, the method of administration used and the like.

The object of the treatment with the pharmaceutical EPO composition of the invention is preferably an increase of the hemoglobin value of more than 6.8 mmol/l in the blood. For this, the pharmaceutical composition may be administered in a way that the hemoglobin value increases between 0.6 mmol/l and 1.6 mmol/l per week. If the hemoglobin value exceeds 8.7 mmol/l, the therapy should be preferably interrupted until the hemoglobin value is below 8.1 mmol/l.

The composition of the invention is preferably used in a formulation suitable for subcutaneous or intravenous or parenteral injection. For this, suitable excipients and carriers are e.g. sodium dihydrogen phosphate, disodium hydrogen phosphate, sodium chlorate, polysorbate 80, HSA and water for injection. The composition may be administered three times a week, preferably two times a week, more preferably once a week, and most preferably every two weeks.

Preferably, the pharmaceutical composition is administered in an amount of 0.01-10 μg/kg body weight of the patient, more preferably 0.1 to 5 μg/kg, 0.1 to 1 μg/kg, or 0.2-0.9 μg/kg, most preferably 0.3-0.7 μg/kg, and most preferred 0.4-0.6 μg/kg body weight.

In general, preferably between 10 μg and 200 μg, preferably between 15 μg and 100 μg are administered per dose.

The invention further relates to the use of a HAS-EPO of the invention for the preparation of a medicament for the treatment of anemic disorders or hematopoietic dysfunction disorders or diseases related hereto.

The invention is further described below by way of examples, which are not to be construed as being in any way limiting to the present invention.

### SHORT DESCRIPTION OF THE FIGURES

FIG. 1 shows HPAEC-PAD analysis native N-linked oligosaccharides obtained from EPO preparations which have undergone removal of sialic acid residues by acid treatment (2 and 3) or not (1 and 4)

1 represents total N-linked oligosaccharides from the EPO preparation F98
2 represents oligosaccharides obtained from EPO F99
3 represents oligosaccharides obtained from the EPO preparation G02
4 represents oligosaccharides obtained from the EPO preparation G04

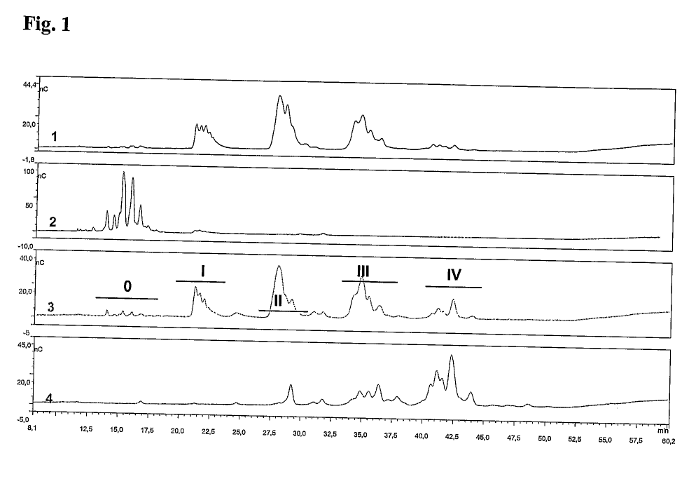

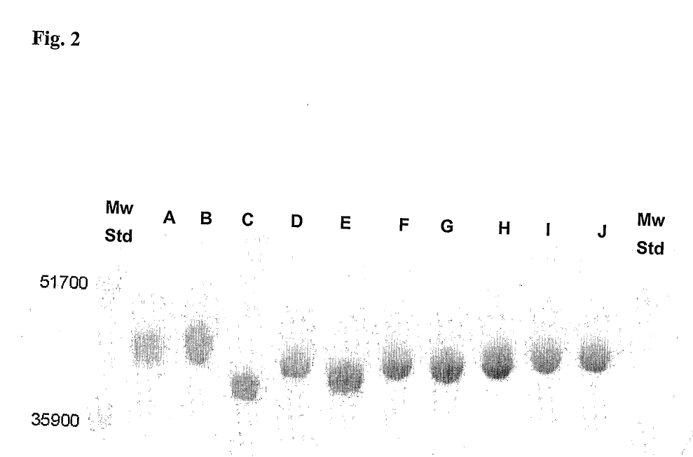

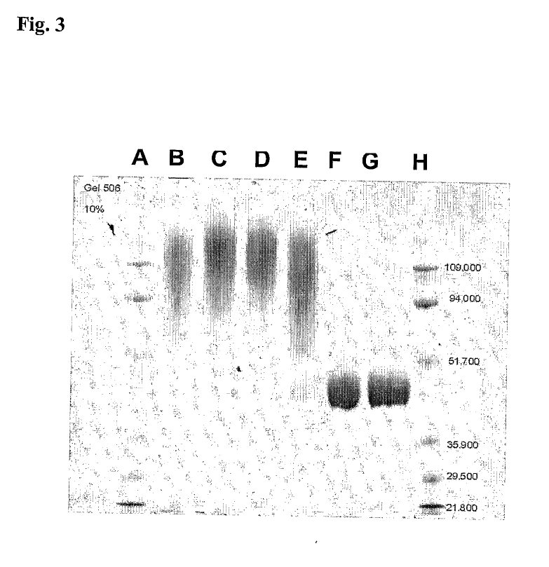

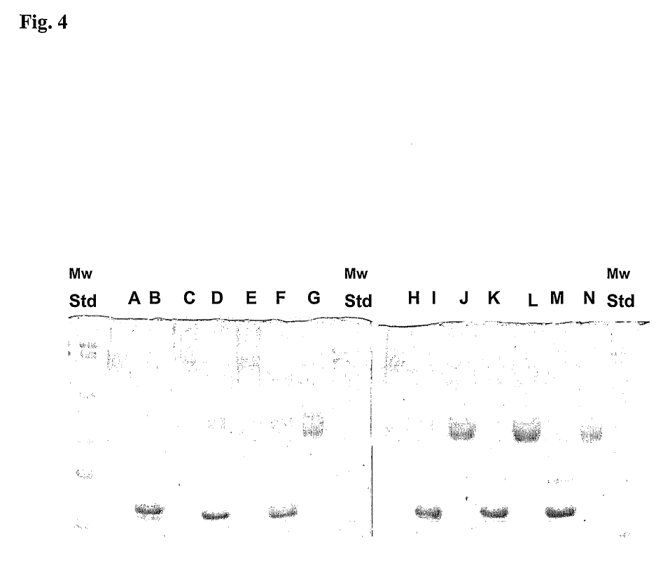

In FIG. 1, the Roman numbers in bold represent the following:

0 represents the neutral oligosaccharide fraction,
I represents the mono-charged oligosaccharide fraction (1 sialic acid),
II represents the di-charged oligosaccharide fraction (2 sialic acid),
III represents the tri-charged oligosaccharide fraction (3 sialic acid),
IV represents the tetra-charged oligosaccharide fraction (4 sialic acid).

FIG. 2 shows an SDS page analysis of different EPO preparations before and after the oxidation by Galox as described in Example B4. For gel electrophoresis a XCell Sure Lock Mini Cell (Invitrogen GmbH, Karlsruhe, D) system was employed. A 10% Bis-Tris gel (NP0301BOX) together with a MOPS SDS running buffer at reducing conditions (both Invitrogen GmbH, Karlsruhe, D) were used according to supplier's instructions.

The lanes represent:

MWStd: Protein marker prestained SDS-PAGE Standards (Bio-RAD, cat. 161-0305, lot 99393); molecular weight marker from top to bottom: 109 kD, 94 kD, 51.7 kD, 35.9 kD, 29.5 kD, 21.8 kD;
A and B: EPO preparation F 98 A after (A) and before (B) Galox;
C and D: EPO preparation F99 after (C) and before (D) Galox;
E and F: EPO preparation G01 after (E) and before (F) Galox;
G and H: EPO preparation G02 after (G) and before (H) Galox;
I and J: EPO preparation G03 after (I) and before (J) Galox

FIG. 3 shows an SDS page analysis of the HES-EPO conjugates prepared according to Example 3. For gel electrophoresis a XCell Sure Lock Mini Cell (Invitrogen GmbH, Karlsruhe, D) system was employed. A 10% Bis-Tris gel (NP0301BOX) together with a MOPS SDS running buffer at reducing conditions (both Invitrogen GmbH, Karlsruhe, D) were used according to suppliers instructions.

The lanes represent:
A: Protein marker prestained SDS-PAGE Standards (Bio-RAD, cat. 161-0305, lot 99393) Molecular weight marker from top to bottom: 109 kD, 94 kD, 51.7 kD, 35.9 kD, 29.5 kD, 21.8 kD,
B: EPO preparation F 98
C: EPO preparation F 99
D: EPO preparation G 01
E: EPO preparation G02
F: EPO preparation G03
G: EPO preparation G04
H: molecular weight standard (see lane A).

FIG. 4 shows an SDS page analysis of Q-Sepharose purified HES-modified EPO preparations F98-G04 before and after removal of N-linked oligosaccharides by polypeptide N-glycosidase. A XCell Sure Lock Mini Cell (Invitrogen GmbH, Karlsruhe, D) system was employed. A 10% Bis-Tris gel (NP0301BOX) together with a MOPS SDS running buffer at reducing conditions (supplied by Invitrogen GmbH, Karlsruhe, D) were used according to suppliers' instructions.

The lanes represent:
Lane MWStd: Protein marker prestained SDS-PAGE Standards (Bio-RAD, cat. 161-0305, lot 99393) Molecular weight from top to bottom: 109 kD, 94 kD, 51.7 kD, 35.9 kD, 29.5 kD, 21.8 kD,
A and B: EPO preparation F 98 before (A) and after (B) treatment with N-glycosidase
C and D: EPO preparation F99 before (C) and (D) treatment with N-glycosidase
E and F: EPO preparation G01 before (E) and after (F) treatment with N-glycosidase
G: EPO BRP standard batch II without treatment with N-glycosidase
H and I: EPO preparation G02 before (H) and after (I) treatment with N-glycosidase
J and K: EPO preparation G03 before (J) and after (K) treatment with N-glycosidase
L and M: EPO preparation G04 before (L) and after (M) treatment with N-glycosidase
N: EPO BRP standard batch II without treatment with N-glycosidase

### EXAMPLES

### A. Materials

### A1. Chemicals

H2SO4 (#K027.1), NaOH (#K021.1), Disodiumhydrogen-phosphate-Dihydrate (#4984.1) and Sodiumdihydrogenphosphate-Dihydrate (#T879.2) were from Carl Roth GmbH Karlsruhe, Germany, Galactose-Oxidase (450 units; #7907) and Catalase (1.4 mg/ml; 55600 units/mg; #C-3556) were from Sigma, L-methionin (#64391) was from Fluka, the protease inhibitors Leupeptin (#1017101), Pepstatin (#253286), Aprotinin (#236624), TLCK (#874485) and Prefabloc SC (#429868) were from Roche.

When Prefabloc SC solution was used, it was prepared immediately before use. The amounts specified below were used to prepare 46 μl protease-inhibitor mix (3 μl Leupeptin 1 mg/ml in water, 150 Pepstatin 1 mg/ml in ethanol, 0.5 μl Aprotinin 2 mg/ml in water, 2.5 μl TLCK 20 mg/ml in water and 25 μl Pefabloc SC 40 mg/ml in water (fresh solution).

### B. Methods

### B1. Procedure for Limited/Complete Acid Hydrolysis of EPO (Glycoproteins) (for the Generation of Unmasked (Terminal) Galactose Residues)

0.5-2.5 mg/ml EPO in low concentration buffer (20 mM Na-phosphate pH 7.0) is heated in a water bath set at 80° C.; in parallel a vial equipped with a calibrated thermometer is incubated; after the temperature has reached 75° C. the sample is brought to 0.1 N H2SO4 with 1N H2SO4. Incubation at this temperature is performed for 0, 4, 10 and 60 minutes after which time samples are neutralized using 1 N NaOH and cooled to 0° C. in a water bath. Samples are either immediately used for the further process steps or are stored at −80° C. after freezing in liquid nitrogen.

### B2. Buffer Exchange Using Vivaspin Concentrator Units

Before the galactose oxidase step EPO-samples are subjected to a buffer exchange to 20 mM Na-phosphate buffer pH7.2 using Vivaspin-concentrator units (10,000 Da molecular weight cut-off (MWCO)). 6 ml or 20 ml volumes are handled according to the manufacturers suggestions with a “Megafuge 1.R” centrifuge (Kendro) at 4000 rpm or similar equipment at 4-8° C. The vivaspin centrifugation step is run at least 3× each by adjusting the protein samples with 20 mM Na-phosphate buffer pH 7.2 before centrifugation. The final EPO samples are concentrated by use of the EUR.PHar method according to SOP-AA-018-01/02.

### B3. Determination of the Sialic Acid Content of Glycoproteins

The method described for the quantitative determination of the sialic acid content of recombinant human EPO is used (Eur. Phar) and is performed according to SOP SOP-AA-052-01/00. No corrections are made for the low sialic acid values expected for EPO subjected to hydrolysis for 60 min. The colorimetric test according to the Eur. Phar. was found to give higher values for the sialic acid contents than HPAEC-PAD mapping, possibly due to a certain unspecificity of the method.

### B4. Galactose-Oxidase Treatment of EPO Samples

### B4.1 Preincubation of Galactose Oxidase

Commercial galactose oxidase may contain proteases which are removable by ion-exchange chromatography. Alternatively, these proteolytic enzymes are inactivated by preincubation in the presence of the above mentioned protease inhibitor mix as follows:

500 μl aliquots of galactose oxidase 400-450 U/ml (according to manufacturer's specification) were mixed with 46 μl of protease inhibitor mix and were incubated at 37° C. for 1 h.

After this incubation step the protease inhibitors were partially removed using vivaspin concentrators (10.000 Da MWCO). The preincubated galactose oxidase was spun down twice with 450 μl of 20 mM Na-phosphate buffer pH 7.2. Subsequently concentrator units were rinsed with 20 mM Na-phosphate buffer pH 7.2 and the solution was then adjusted to 450 units of galactose oxidase/400 μl. For the subsequent step, 23.5 μl of galactose oxidase (450 units/400 μl) (=26.4 units) in 20 mM Na-phosphate buffer pH 7.2 were used per ml of EPO sample.

### B4.2 Galactose-Oxidase Reaction

EPO samples were adjusted to a final concentration of 10 mM methionine (for the protection against polypeptide oxidation), 2.35 μl of catalase (6200 units/200 μl) and 23.5 preincubated galactose oxidase were added and incubated at 37° C. for 12-18 hours. After incubation aliquots of the samples were taken for SDS-PAGE analysis. The samples were then subjected to ion-exchange chromatography.

### B5 Ion Exchange Chromatography for the Purification of EPO and EPO Derivatives:

The purification of EPO samples was performed at room temperature using an ALTA explorer 10 system (Amersham Pharmacia Biotech) consisting of a pump P-903, a Mixer M-925, with 0.6 ml chamber, a Monitor UV-900, with 10 mm flow cell, a Monitor pH/C-900, a fraction Collector Frac-900, a sample loop 2 ml along with the Unicorn software Version 3.21. The column containing 5 ml Q-Sepharose Fast Flow was equilibrated with 10 CV of buffer A (20 mM N-morpholino-propane sulfonic acid/NaOH buffer, pH 8.0). The EPO samples were diluted 1:10 with buffer A and finally adjusted to pH 7.8-8.2 and were applied by using the sample pump at a flow rate of 1 ml/min. Following washing of the sample pump with 10 ml of buffer A, the column was further washed with 6 CV of buffer A at a flow rate of 1.0 ml/min. Subsequently a 4 volumes of 20 mM Na-Phosphate, pH 6.5; buffer B at a flow rate of 0.8 ml/min and EPO was eluted using a steep gradient from 0-40% buffer C (0.5M NaCl in 20 mM Na-Phosphate, pH 6.5) within 37 min. Elution profiles were recorded at 206, 260 and 280 nm absorbance. After completion of elution, the column was regenerated by 25 ml of buffer C at a flow rate of 1 ml/min. Finally the column was run with 1M NaOH for 60 min and reconditioned with buffer C and stored until further use. 3-4 pools of the EPO containing fractions were analysed by SDS-PAGE.

B 6. Removal of Buffer Salts from EPO after Ion Exchange Chromatography

For EPO concentration and buffer exchange for the subsequent HES-modification reaction, the EPO samples obtained after ion-exchange chromatography were subjected to a Vivaspin desalting procedure similar as described under paragraph B2. EPO samples were concentrated to 3-5 mg/ml final protein concentration and were diluted three times with 4 volumes of 0.1M Na-Acetate pH 5.5 and reconcentrated after each dilution step using Vivaspin concentrators (6 ml or 20 ml concentrator units) at 4000 rpm. The protein was then removed from the concentrator units and adjusted to 2-4 mg/ml. Finally the resulting protein concentration was determined according to Eur. Phar (OD 280 nm). The protein was used for HES-modification within 24 hours and until then stored in ice/water at 0° C. (Sterile filtration may be required for storage of samples for longer time periods, alternatively samples can be stored frozen at −80° C. after freezing in liquid nitrogen after adjusting to pH 7.2 in PBS).

### B7 Synthesis of HydroxylaminoHES50/0.7

5 g of HES50/0.7 (Lot. 304, MW=47000 D, DS=0.76, Supramol Parenteral Colloids GmbH, Rosbach-Rodheim, D) were dissolved in 40 ml 0.1M sodium acetate buffer, pH 5.2 and 10 mmol O-[2-(2-aminooxy-ethoxy)-ethyl]-hydroxylamine were added. After shaking for 26 h at 22° C., the reaction mixture was slowly added to 200 ml of an ice-cold 1:1 mixture of acetone and ethanol (v/v). The precipitated product was collected by centrifugation at 0° C., washed with 30 ml of an ice-cold 1:1 mixture of acetone and ethanol (v/v), re-dissolved in 50 ml water, dialysed for 2 d against water (SnakeSkin dialysis tubing, 3.5 kD cut off, Perbio Sciences Deutschland GmbH, Bonn, D) and lyophilized. The yield of isolated product was 79%.

### B8 Conjugation of HydroxylaminoHES50/0.7 to EPO

To 2.45 ml of a solution of oxidized EPO of step B6 in 0.1 M sodium acetate buffer, pH 5.5 (1.633 mg/ml), 2.45 ml of a solution of 333 mg of HydroxylaminoHES50/0.7 (from step B7) in the same buffer were added and the mixture was shaken gently for 23.5 h at 22° C. The reaction mixture was then purified by FPLC and analyzed by gel electrophoresis.

### B9. Buffer Exchange Using Vivaspin Concentrators

HES-modified EPO and EPO from appropriate control incubations were subjected to buffer exchange by using 5 ml Vivaspin concentrators (10,000 Da MWCO) and centrifugation at 4000 rpm at 6° C. as described above. Samples (1-5 mg of EPO protein) were concentrated to 0.5-1.5 ml and were diluted with phosphate buffered saline (PBS) pH 7.1+/−0.2 to 5 ml and subjected to 10-fold concentration by centrifugation. Each sample was subjected to the concentration and dilution cycle three times. Finally, samples were withdrawn and the concentrator units were washed with 2×0.5 ml of PBS. Samples were frozen in liquid nitrogen at protein concentrations of approximately 1.2 mg/ml. Protein concentration in the final EPO solutions was determined by measuring the absorbance at 280 nm using the specific absorbance value of 7.43 as described in the European Pharmacopeia (Erythropoietin Concentrated Solution, 4th Edition, 2002, pages 1123-1128). Alternatively protein concentration was also determined by RP-HPLC using the International BRP batch II reference EPO preparation as a standard.

B10. Liberation of N-Linked Oligosaccharides with Recombinant Polypeptide N-Glycosidase (Roche, Penzberg, Germany)

To 200-600 μg aliquots of native, complete or partially desialylated or galactose oxidase treated EPO samples in 50 mM Na-phosphate buffer pH 7.2 25 μl of recombinant polypeptide N-glycosidase were added (Roche, Penzberg, Germany; 250 units/250 μl). The reaction mixture was incubated at 37° C. for 12-24 hours. Occasionally 5-10 μl of polypeptide N-glycosidase was added after 12-14 h after start of the incubation.

The release of N-glycosidically bound oligosaccharides was checked by SDS-PAGE analysis of 5-10 μg protein under reducing conditions and subsequent staining of protein bands with Coomassie Blue (Carl Roth GmbH, Karlsruhe, Germany), which detected the specific shift of the EPO protein band to the migration position of the de-N-glycosylated EPO forms.

B11. Separation of N-Linked Oligosaccharides and HES-Modified Glycans from De-N-Glycosylated EPO Protein by RP-HPLC

Separation of all de-N-glycosylated EPO samples from HES-modified and unmodified EPO protein samples was performed at room temperature using an HPLC-system supplied by Dionex GmbH (Idstein, Germany) consisting of a P 680 A HPG pump, a Degasys DG 1210 degassing system, an autosampler (Automated Sample Injector ASI-100), a sample loop of 250 μl, a column thermostatter department TCC 100 along with a UV/Vis-Detektor UVD170U. For larger amounts of protein (>1 mg) an ÄKTA explorer 10 system equipped with a Pump P-903, Mixer M-925 with 0.6 ml chamber, Monitor pH/C-900, pump P-950 (sample pump) along with a Software Unicorn Version 3.21 was used. Detection of peaks was at 280, 220 and 206 nm using a Monitor UV-900 with a 10 mm flow cell.

Aliquots of PNGase digests of 0.1-0.6 mg of HESylated EPO were applied to a EC 250/4.6 Nucleosil 120-5 C4 RP column (Macherey-Nagel, Germany Cat. Nr. 720096.46) equipped with a precolumn, e.g. CC 8/4 Nucleosil 120-5 C4, Macherey-Nagel, Kat. Nr. 721889.40.

The column was equilibrated with 2-4 CV of 5% eluent B (0.1% TFA, 90% acetonitrile). 250 μl samples of de-N-glycosylated EPO forms were then injected and the sample loop was washed with 8 ml of 5% eluent B. Following washing of the column with 0.2 CV of 5% eluent B, a linear gradient from 5% to 50% eluent B over 18 min was applied. Elution of the column was continued by a gradient to 66% eluent B (over 20 min) after which time 100% of eluent B was applied over 3 min and the column was washed with 100% eluent B for further 5 min. Fractions were collected every 1 min (1 ml).

Unmodified oligosaccharides were recovered from the flow through and, in the case of HESylated EPO, from fractions eluting at a concentration of about 25% eluent B. conteined HESylated N-glycans. The protein eluted in a volume of 4-6 ml at a concentration of 53% eluent B.

Fraction containing oligosaccharides were neutralized and were concentrated in a speed-vac concentrator or were lyophilized. The glycan samples were desalted using HyperCarb cartridges (100 or 200 mg) as follows: prior to use, the cartridges were washed three times with 500 μl 80% (v/v) acetonitrile in 0.1% (v/v) TFA followed by three washes with 500 μl water. The samples were diluted with water to a final volume of at least 300 μl before applying to the cartridges. They were rigorously washed with water. Oligosaccharides were eluted with 1.2 ml 25% acetonitrile containing 0.1% (v/v) TFA. The eluted oligosaccharides were neutralised with 2 M NH₄OH and were dried in a Speed Vac concentrator. They were stored at −80° C. in H₂O until further use.

HES-modified N-glycans were neutralized and dried in a speed vac concentrator or were lyophilized, they were desalted using VIVaspin concentrator units (5,000 kDa cut-off) and the material was dissolved in 200-500 μl of water and stored until further analytical use.

### Results

### 1 Acid Treatment of Fully Sialylated EPO Samples for the Removal of Terminal Sialic Acid Residues

Samples of pharmaceutical grade EPO was treated as described in Example B1, and the reaction stopped at various timepoints. Sample F48a-II (yielding final product F99) was treated for 60 min, sample F48a-III for 10 min acid treatment (G01), sample F48a-IV for 4 min (G02), sample F48a-V for 0 min (G03) and sample F48a-VI was not subjected to acid treatment to serve as a control (G04). After buffer exchange as described in Example B2, aliquots of the acid treated EPO samples were analyzed as described in B 10 and compared with sample GT012a-I, a undersialylated side fraction from a recombinant EPO preparation with very low in vivo bioactivity. As shown in FIG. 2, acid treatment leads to a shift in molecular weight (compare lanes C with D and lanes E with F), indicating that acid treatment is efficient in removing terminal sialic acid residues.

2 RP-HPLC Separation of Liberated N-Glycans from De-N-Glycosylated EPO Polypeptide

The above mentioned aliquots were subjected to buffer exchange as described in B9 and PNGase treatment as described in B10. The de-N-glycosylated EPO forms were separated from liberated N-glycans by RP-HPLC (as described in Example B11) and the resulting oligosaccharide fractions were subjected to further analysis. The oligosaccharide fractions obtained after RP-HPLC of PNGase-treated EPO forms were desalted and aliquots corresponding to 0.5-3 nmoles were subjected to HPAEC-PAD analysis as described under Example B11. Mapping of the N-glycans of untreated EPO yielded an oligosaccharide pattern as depicted in FIG. 1, panel 4. Based on peak response in HPAEC-PAD 0.5% of the oligosaccharide peak area was detected in the region of non-sialylated glycans (12.5-18 min), 1.3% of the oligosaccharide peak area was detected in the region of monosialylated glycans (21-25 min), 11.3% disialylated (26-30.5 min), 28.1% trisialylated (33-38 min) and 58.7% tetrasialylated glycans (39-45 min).

In the F98 sample (panel 1 in FIG. 1), the resulting N-glycans eluted as follows: 1.5% in the non-sialo, 18.8% in the monosialo, 41.2% in the disialo, 32.2% in the trisialo and only 6.4% in the tetrasialo region.

These values are very similar to the sample, which was subjected to acid-mediated sialic acid removal for four minutes (G02, panel 3 in FIG. 1). In the F98 sample, the resulting N-glycans eluted as follows: 2.8% in the non-sialo, 17.5% in the monosialo, 34.9% in the disialo, 31.6% in the trisialo and 13.1% in the tetrasialo region.

In comparison thereto, acid-mediated sialic acid removal for 60 minutes removes the terminal sialic acid residues almost completely (panel 2 of FIG. 1). In the F99 sample, the resulting N-glycans eluted as follows: 93% in the non-sialo, 4.8% in the monosialo, 2.3% in the disialo region. The signals in the in the trisialo and tetrasialo regions were negligible.

The values obtained for the sialic acid content of the samples by the above-described HPAEC-PAD analysis deviate somewhat from the value obtained with the Phar. Eur. standard method. Values obtained with this method are given in the table below:

TABLE
Sialic acid determination of EPO preparations for
subsequent galactose oxidase and HES modification

| | Final sample | nMol | |
|---|---|---|---|
| | name | sialic | |
| | after sterile | acid/ | |
| Sample name: | filtration | nmol EPO | Description of sample |
| GT012a-I-1.2 | F98 | 6.0 | Untreated GT012 |
| 0412-22/F48a-II-1.2 | F99 | 0.5 | 60 min acid hydrolysis |
| 0412-22/F48a-III-1.2 | G01 | 3.0 | 10 min acid hydrolysis |
| 0412-22/F48a-IV-1.2 | G02 | 7.2 | 4 min acid hydrolysis |
| 0412-22/F48a-V-1.2 | G03 | 12 | 0 min acid hydrolysis |
| 0412-22/F48a-VI-1.2 | G04 | 12 | Untreated |

It is interesting that the untreated sample GT0012a-I shows a high degree of undersialylated glycoprotein forms even without a prior acid treatment (panel 4). This means that this form might contain terminal galactose residues instead of terminal sialic acid residues.

### 3 Galactose Oxidase Treatment of EPO Samples in Preparation for HESylation

HESylated galactose oxidized EPO fractions (after step B10) were analyzed by way of comparative SDS-PAGE analysis of purified and sterile filtrated final EPO preparations.

As can be seen from FIG. 3, samples B, C, D, and E were efficiently HESylated. They have only a slightly different molecular weight distribution in SDS-PAGE due to their different sialic acid content apart from the covalently HES modification. In sample E a faint band corresponding to minute amounts of non-modified EPO is visible The fully sialylated EPO forms G03 and G04 (lanes F and G) are not HES modified. Samples F98 and G02 have some fully (tetrasialylated) N-glycans and a small proportion of these EPO preparations are assumed to lack HES modification at one (or two) N-glycosylation sites resulting in HES modified forms of lower molecular weight compared to F99 and G01.

### 4 HESylation of Modified EPO Forms at N-Glycans.

EPO fractions obtained after galactose oxidase treatment and subsequent buffer exchange (step B6) were conjugated to HydroxylaminoHES50/0.7 (step B8) and prepared for further analysis by way of a buffer exchange (step B9). In FIG. 4, it was confirmed that HES-modification occurred at N-glycans. Aliquots of samples were subjected to de-N-glycosylation by polypeptide N-glycosidase (PNGase) treatment (see Example B10). 5-10 μg aliquots of samples before and after PNGase treatment were subjected to SDS-PAGE analysis. As is depicted in FIG. 4, the samples F98, F99, G01, G02, G03 and G04 yielded the O-glycosylated EPO form after incubation with PNGase. Variations in SDS-PAGE patterns of de-N-glycosylated EPO forms are due to different degree of desialylation of the O-glycan moiety in F98, F99, G01-G04.

In conclusion, successful HESylation of EPO, oxidized with galactose oxidase, was possible, even in case of the sample F98, which had not been previously treated by an acid step to remove terminal sialic acid residues. Thus, this EPO-sample—an EPO preparation having a lower than normal sialic acid content—was found to contain significant amounts of terminally exposed galactose residues giving free aldehyde groups after treatment by galactose oxidase available for conjugation with an amino-group-containing HAS. The in vivo data gave a value for the specific activity of this conjugate of about 200 000 U/mg and thus even higher than the fully sialylated form of EPO (120 000-130 000 U/mg). It was thus possible to convert a “junk” fraction of EPO into a highly active protein suitable for therapeutic use.

1-74. (canceled)

75. A hydroxyalkylstarch (HAS)-glycoprotein(GPO)-conjugate (HAS-GPO), comprising one or more HAS molecules, wherein each HAS is conjugated to the GPO via a galactose moiety of an N- or O-glycan of the glycoprotein.

76. The HAS-GPO of claim 75, wherein the GPO is selected from the group consisting of erythropoietin (EPO), IFN beta, GM-CSF, APC, tPA, A1AT, AT III, HCG, LH, FSH, an antibody fusion protein, a therapeutic antibody, an interleukin, IFN-[alpha], CSF, factor VII, factor VIII, and factor IX.

77. The HAS-GPO of claim 75, wherein essentially all asparagines and glutamine side chains of the GPO retain an intact amido group.

78. The HAS-GPO of claim 75, wherein essentially none of the methionine side chains of the GPO is in an oxidized form.

79. The HAS-GPO of claim 75, wherein the GPO comprises at least one N-glycan or O-glycan comprising at least one terminal galactose moiety.

80. The HAS-GPO of claim 79, wherein the GPO is EPO comprising two or three N-glycans each comprising at least two terminal galactose moieties.

81. The HAS-GPO of claim 75, wherein the GPO was derived from mammalian, insect, yeast cells or genetically engineered calls.

82. The HAS-GPO of claim 75, wherein HAS is conjugated to the GPO via a linker molecule.

83. The HAS-GPO of claim 75, comprising 1-12 HAS molecules per GPO molecule.

84. (canceled)

85. The HAS-GPO of claim 75, wherein the HAS is hydroxyethylstarch (HES) and wherein the HES has a molecular weight of 1 to 300 kDa and exhibits a molar degree of substitution of 0.1 to 0.8 and a ratio between C₂:C₆-substitution in the range of 2-20, with respect to the hydroxyethyl groups.

86-87. (canceled)

88. A method for the production of a HAS-GPO according to claim 75, comprising the steps of:
a) providing a GPO comprising at least two terminal sugar moieties which are galactose residues,
b) oxidizing terminal galactose residues by the action of the enzyme galactose oxidase to form terminal galactose residues comprising a reactive aldehydro group,
c) providing modified HAS being capable of reacting with the GPO of step b), and
d) reacting the GPO of step b) with the HAS of step c), whereby an HAS-GPO is produced comprising one or more HAS molecules, wherein each HAS is conjugated to the GPO via a galactose moiety of an N-glycan or O-glycan of the glycoprotein, and wherein said N-glycan or O-glycan further comprises at least one terminal sugar moiety which is not a sialic acid residue.

89. The method of claim 88, wherein the GPO is EPO.

90. The method of claim 88, wherein the GPO is always kept at a pH between 3.0 and 9.0, prior to and during the method of claim 88, whereby a HAS-GPO is produced wherein essentially all asparagines and glutamine side chains of the GPO retain an intact amido group.

91. The method of claim 88, wherein the GPO was derived from mammalian, insect or yeast cells or genetically engineered cells.

92. The method of claim 90, wherein the terminal galactose unit is oxidized after partial or complete enzymatic removal of the terminal sialic acid.

93. The method of claim 91, wherein in step d) the modified HAS is conjugated to the oxidized terminal saccharide unit.

94. The method of claim 88, wherein the HAS of step c) of claim 88 is modified such that it comprises a free hydrazide, hydroxylamine, or semicarbazide function.

95. The method of claim 88, wherein step d) is performed in a reaction medium comprising at least 10% per weight H₂O.

96. The method of claim 88, wherein the HAS is conjugated to the GPO via a linker molecule.

97. The method of claim 88, wherein the HAS is HES having a molecular weight of 1 to 300 kDa and exhibiting a molar degree of substitution of 0.1 to 0.8 and a ratio between C2:C6-substitution in the range of 2-20, with respect to the hydroxyethyl groups.

98. (canceled)

99. A HAS-GPO, as obtainable by the method of claim 88.

100-101. (canceled)

102. A pharmaceutical composition comprising the HAS-GPO according to claim 75.

103. The pharmaceutical composition of claim 102, further comprising at least one pharmaceutically acceptable carrier, wherein the HAS-GPO constitutes at least 5% of the total protein present in the pharmaceutical composition, wherein the HAS-GPO is present in a concentration above 1 nM, and wherein the HAS-GPO is HAS-EPO.

104. A method for treating an anemic disorder or a hematopoietic dysfunction disorder in a subject, comprising administering to said subject a composition comprising the HAS-GPO according to claim 75, wherein said HAS-GPO is HAS-EPO.

105-115. (canceled)

116. The HAS-GPO of claim 75, wherein the N- or O-glycan further comprises at least one terminal sugar moiety which is not a sialic acid residue.
